(19) United States
(12) Patent Application Publication
(10) Pub. No.: US 2024/0384229 A1
(43) Pub. Date: Nov. 21, 2024

(54) METHOD FOR PROVIDING A CELL POPULATION ENRICHED IN NEURONS AND PRECURSORS THEREOF

(71) Applicant: Novo Nordisk A/S, Bagsvaerd (DK)

(72) Inventors: Jonathan Niclis, Koebenhavn (DK); Josefine Raagaard Christiansen, Copenhagen N (DK)

(21) Appl. No.: 18/576,838

(22) PCT Filed: Jul. 13, 2022
(86) PCT No.: PCT/EP2022/069565

§ 371 Date: Jan. 5, 2024

(30) Foreign Application Priority Data
Jul. 14, 2021 (EP) 21185527.5

(51) Int. Cl. C12N 5/0793; C12N 5/00

(52) U.S. Cl. CPC C12N 5/0619; C12N 5/0087; C12N 2501/119; C12N 2501/13; C12N 2506/02

(57) ABSTRACT

The present invention relates to a method for providing a cell population enriched in neurons and/or precursors thereof, comprising the steps of obtaining a cell population comprising neuron precursors, and selecting cells positive indicative of whether a cell has high propensity for developing into a neuron.

### TECHNICAL FIELD

The present invention relates generally to the field of stem cells, such as human pluripotent stem cells (hPSCs). Specifically, methods are provided for enriching a cell population in stem cell-derived neurons and precursors thereof.

### BACKGROUND

Human pluripotent stem cells have the potential to revolutionize the treatment of various intractable diseases and disorders of the human body. Treatments include cell-replacement therapy of neural conditions such as Parkinson's disease. For such treatments to become viable, however, it requires the development of in vitro methods to artificially produce stem cell-derived products for their delivery to the central nervous system (CNS).

The differentiation of hPSCs into defined cell types is a difficult process to control; in many cases the progeny generated from a specific protocol is heterogeneous and comprise a mixture of cell types. Presently, several methods are described to differentiate hPSCs to neural stem cells and subsequently into neurons. However, all protocols generate heterogenous populations, i.e. not all cells are necessarily neuron progenitors and may specialize into a different cell type, or the asynchronous nature of in vitro differentiation and in vivo development results in different cell stages present at the same time. Heterogeneous cultures are sub-optimal for investigating disease and normal biology in a single/specific cell type of interest, or when transplantation of a single/specific cell type is required, such as in the context of cell replacement therapy and specifically where neurons are the intended therapeutic cell type. The transplantation of mixed populations of cells reduces the safety and efficacy profile of a cell therapy and increases the risks of undesirable side-effects from undesirable cell types. Methods for separating a cell population to obtain a higher purity of neurons and precursors thereof has been described in WO 2021/042027. However, there is still a need for further methods to obtain enriched cell populations comprising neural cells having an increased propensity for developing into neurons.

It is therefore an object of the present invention to improve the homogeneity of a cell population comprising neural cells towards cell types determined to develop into neurons and precursors thereof, such as ventral midbrain neurons.

### SUMMARY

The object as outlined above is achieved by the aspects of the present invention. In addition, the present invention may also solve further problems, which will be apparent from the disclosure of the exemplary embodiments.

A first aspect of the present invention relates to a method for providing a cell population enriched in neurons and/or precursors thereof, such as intermediate precursor cells, comprising the steps of obtaining a cell population comprising neuron precursors, selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

The present inventors have found it possible to increase in a cell population the proportion of cells having propensity for developing into neurons by isolating specific cells, wherein the specific cells are isolated by positively and/or negatively selecting on certain surface markers. Furthermore, the present inventors have found that the timing of selecting and/or excluding cells may improve the outcome, in particular when applied to a cell population being differentiated into neural cells such as neurons. Specifically, a cell developing into a neuron, such as in an in vitro differentiation process, progresses through different stages; initially the cell becomes a neural stem cell, which then further differentiates into an intermediate precursor cell before turning into a neuron. The present inventors found that the specific surface markers indicative of whether or not a neural stem cell has neuronal fate are reliably expressed only once the neural stem cell further develops. In a cell population undergoing differentiation into neuron identity the individual cells will be at different stages of development and the cell population may comprise subpopulations of cells, such as pluripotent stem cells, neural stem cells, intermediate precursor cells, and neurons. Furthermore, individual cells transitioning from one stage to another may express markers indicative of both stages. The present inventors found that the selection and/or exclusion of cells is preferably applied to a cell population, wherein at least part of the cells have further developed from being neural stem cells. The expression marker SOX2 is characteristic of neural stem cell identity. As the neural stem cell further develops the cell at some point no longer expresses SOX2. This coincides with specific surface markers being expressed or silenced on the cells having neuronal fate. Accordingly, a decrease in expression of SOX2 in a cell population is indicative of neural stem cells further developing and may be used to identify the window of opportunity for isolating the cells having propensity for developing into neurons.

Therefore, an aspect of the present invention relates to method for isolating neurons and/or precursors thereof from a cell population, comprising the steps of obtaining a cell population comprising neural stem cells expressing the marker SOX2, further culturing the cell population until the expression of SOX2 in the cell population decreases, selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

Alternatively, the timing of selecting and/or excluding cells may also be established based on markers indicative of part of the neural stem cells having further developed into intermediate precursor cells and/or neurons. In an embodiment, the cell population is obtained by initially differentiating pluripotent stem cells (PSCs) into neural stem cells, and the selection of cells may be applied as the cells have progressed to a certain stage in the differentiation at which the cultures are comprised of uni-potent and/or multi-potent cells such as intermediate precursor cells and/or terminally differentiated cells such as neurons. Therefore, in an embodiment, at least 5% of the cell population express markers of intermediate precursor identity when selecting and/or excluding cells. In an embodiment, at least 5% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1 when selecting and/or excluding cells. The present inventors have identified the expression of these markers as strong indicators of the cell population being matured to a stage, at which positive and/or negative selection of cells having strong propensity for neuronal development can be carried out based on the identified surface markers. Cells prior to having developed into intermediate precursor cells may not sufficiently express or silence the identified surface markers for isolating the cells to be carried out.

The present inventors found that selecting and/or excluding cells to increase neurons in a cell population based on the identified surface markers can be applied to all regions of the brain. In particular, it was found that excluding cells expressing the surface marker CD47 increases the proportion of neurons in a cell population directed towards becoming ventral midbrain neural cells. Furthermore, it was found that excluding cells expressing the surface marker CD99 increases the proportion of neurons in a cell population directed towards forebrain, hindbrain, or spinal cord.

In another aspect of the present invention is provided a cell population of neurons and/or precursors thereof, wherein at least 50% of the cells are positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or less than 50% of the cells are positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1. A cell population according to the above features an increased propensity for developing into neurons.

Another aspect relates to a composition comprising a cell population according to the present invention. In a further aspect is disclosed a cell population or composition according to the present invention for use as a medicament, such as in the treatment of a neurological condition. It is believed that the transplantation of homogeneous populations of cells increases the safety and efficacy profile of the cell therapy and decreases the risks of undesirable side-effects from undesirable cell types. A treatment with a cell population or composition according to the present invention provides an increased ratio of cells with neurons and/or precursors thereof, thereby making it highly suitable for prevention or treatment of a condition requiring the administration of such cells.

### BRIEF DESCRIPTION OF DRAWINGS

FIG. 1 shows a simplified schematic illustrating the stages of differentiation from hPSCs (A) to early NSCs (B) to heterogenous intermediate stage cultures (C) and finally to terminally differentiated cell types (D). The time window at which scRNA-seq analysis was performed is marked with a box (E).

FIG. 2A shows a venn diagram obtained by scRNA-seq analysis of hESCs. The diagram displays the percentage of cells expressing one or more of the markers POU5F1, SOX2, ASCL1, and INA.

FIG. 2B shows t-SNE plots obtained by scRNA-seq analysis of hESCs. The plots show the expression profiles of the genes POU5F1, SOX2, ASCL1, and INA. The expression levels are represented as UMIs per transcript (UTP) and displayed using a colour gradient from no expression (very light grey) to high expression (dark grey).

FIG. 3A shows a venn diagram obtained by scRNA-seq analysis of DIV16 ventral midbrain NSCs. The diagram displays the percentage of cells expressing one or more of the markers POU5F1, SOX2, ASCL1, and INA.

FIG. 3B shows t-SNE plots obtained by scRNA-seq analysis of DIV16 ventral midbrain NSCs. The plots show the expression profiles of the genes POU5F1, SOX2, ASCL1, and INA. The expression levels are represented as UMIs per transcript (UTP) and displayed using a colour gradient from no expression (very light grey) to high expression (dark grey).

FIG. 4A shows a venn diagram obtained by scRNA-seq analysis of DIV24 ventral midbrain neural cells. The diagram displays the percentage of cells expressing one or more of the markers POU5F1, SOX2, ASCL1, and INA.

FIG. 4B shows t-SNE plots obtained by scRNA-seq analysis of DIV24 ventral midbrain neural cells. The plots show the expression profiles of the genes POU5F1, SOX2, ASCL1, and INA. The expression levels are represented as UMIs per transcript (UTP) and displayed using a colour gradient from no expression (very light grey) to high expression (dark grey).

FIG. 5 shows integrated UMAP plots obtained by scRNA-seq analysis DIV22, 24 and 26 ventral midbrain neural cells. The plots show the expression profiles of the genes LMX1A, SOX2, ASCL1, and INA. LMX1A negative cells are marked with circles. The expression levels are represented as UMIs per transcript (UTP) and displayed using a colour gradient from no expression (very light grey) to high expression (dark grey).

FIG. 6 shows integrated UMAP plots obtained by scRNA-seq analysis DIV22, 24 and 26 ventral midbrain neural cells. The plots show the expression profiles of the genes ADGRG1, EPHB2, GAP43, ADCYAP1, C1QL1, and NRXN1. The expression levels are represented as UMIs per transcript (UTP) and displayed using a colour gradient from no expression (very light grey) to high expression (dark grey).

FIG. 7 shows integrated UMAP plots obtained by scRNA-seq analysis DIV22, 24 and 26 ventral midbrain neural cells. The plots show the expression profiles of the genes FZD1, CACNA2D1, DDC, GPR85, and LRRC4C. The expression levels are represented as UMIs per transcript (UTP) and displayed using a colour gradient from no expression (very light grey) to high expression (dark grey).

FIG. 8 shows integrated UMAP plots obtained by scRNA-seq analysis DIV22, 24 and 26 ventral midbrain neural cells. The plots show the expression profiles of the genes TNFRSF21, TRPM8, FREM2, DLL3, DLL1, CHRNA5, and CLDN5. The expression levels are represented as UMIs per transcript (UTP) and displayed using a colour gradient from no expression (very light grey) to high expression (dark grey).

FIG. 9 shows integrated UMAP plots obtained by scRNA-seq analysis DIV22, 24 and 26 ventral midbrain neural cells. The plots show the expression profiles of the genes SLIT2, NTN1, CMTM7, CMTM8, CD36, and CD47. The expression levels are represented as UMIs per transcript (UTP) and displayed using a colour gradient from no expression (very light grey) to high expression (dark grey).

FIG. 10 shows integrated UMAP plots obtained by scRNA-seq analysis DIV22, 24 and 26 ventral midbrain neural cells. The plots show the expression profiles of the genes CD99, TNFRSF1A, TMEM123, LRP10, HLA-A, and RAMP1. The expression levels are represented as UMIs per transcript (UTP) and displayed using a colour gradient from no expression (very light grey) to high expression (dark grey).

FIG. 11 shows integrated UMAP plots obtained by scRNA-seq analysis DIV22, 24 and 26 ventral midbrain neural cells. The plots show the expression profiles of the genes CMTM6, TNFRSF12A, CRLF1, ITGB8, and CD83. The expression levels are represented as UMIs per transcript (UTP) and displayed using a colour gradient from no expression (very light grey) to high expression (dark grey).

FIG. 12 outlines the fluorescence-activated cell sorting (FACS) strategy for obtaining populations enriched in CD47 positive or CD99 negative cells. The figure shows the hierarchical gates, firstly, (A) to select events representing cells and exclude debris, secondly, (B) to select single cells and exclude doublets, thirdly, (C) to select live cells and exclude dead cells, and, finally, (D) to select CD47 positive or CD47 negative populations.

FIG. 13 shows post-sort purity immediately after FACS. The purity of the CD47 positive (A) and CD47 negative (B) cells was assessed by re-analyzing the samples on the flow cytometer.

FIG. 14 shows the percentage of cells expressing CD47 in the sorted CD47 positive (A) and CD47 negative (B) samples, which were analyzed by intracellular flow cytometry immediately after FACS.

FIG. 15 shows the percentage of cells expressing the neural stem cell marker SOX2 in the sorted CD47 positive (A) and negative (B) samples, which were analyzed by intracellular flow cytometry immediately after FACS.

FIG. 16 shows the percentage of cells expressing the proliferation marker Ki67 in the sorted CD47 positive (A) and negative (B) samples, which were analyzed by intracellular flow cytometry immediately after FACS.

FIG. 17 shows the percentage of cells expressing the midbrain floorplate marker FOXA2 in the sorted CD47 positive (A) and negative (B) samples, which were analyzed by intracellular flow cytometry immediately after FACS.

FIG. 18 shows a bar graph summarizing the flow cytometry results obtained by intracellular flow cytometry immediately after FACS of CD47 positive and negative cells.

FIG. 19 shows the morphology of the cells in low (A, B, C) and high (Ai, Bi, Ci) magnification phase-contrast images acquired 24 hours post-FACS of the sorted CD47 positive cells (A, Ai), CD47 negative cells (B, Bi), and “pass-through” control cells (C, Ci). Scale bar: 50 μm.

FIG. 20 shows the morphology of the cells in low (A, B, C) and high (Ai, Bi, Ci) magnification phase-contrast images acquired 5 days post-FACS of the sorted CD47 positive cells (A, Ai), CD47 negative cells (B, Bi), and “pass-through” control cells (C, Ci). Scale bar: 50 μm.

FIG. 21 shows representative immunofluorescence images of sorted CD47 positive (A), CD47 negative (B), and “pass-through” control (C) cells stained for Ki67 and DAPI at 5 days post-FACS. Scale bar: 50 μm.

FIG. 22 shows representative immunofluorescence images of sorted CD47 positive (A), CD47 negative (B), and “pass-through” control (C) cells stained for FOXA2 and DAPI at 5 days post-FACS. Scale bar: 50 μm.

FIG. 23 shows representative immunofluorescence images of sorted CD47 positive (A), CD47 negative (B), and “pass-through” control (C) cells stained for TH and DAPI at 5 days post-FACS. Scale bar: 50 μm.

FIG. 24 outlines the fluorescence-activated cell sorting (FACS) strategy for obtaining populations enriched in CD99 positive or CD99 negative cells. The figure shows the hierarchical gates, firstly, (A) to select events representing cells and exclude debris, secondly, (B) to select single cells and exclude doublets, thirdly, (C) to select live cells and exclude dead cells, and, finally, (D) to select CD99 positive or CD99 negative populations.

FIG. 25 shows post-sort purity immediately after FACS. The purity of the CD99 positive (A) and CD47 negative (B) cells was assessed by re-analyzing the samples on the flow cytometer.

FIG. 26 shows post-FACS viability of cells sorted based on CD99 expression.

FIG. 27 shows the morphology of the cells in low (A, B, C) and high (Ai, Bi, Ci) magnification phase-contrast images acquired 24 hours post-FACS of the sorted CD99 positive cells (A, Ai), CD99 negative cells (B, Bi), and “pass-through” control cells (C, Ci). Scale bar: 50 μm.

FIG. 28 shows the morphology of the cells in low (A, B, C) and high (Ai, Bi, Ci) magnification phase-contrast images acquired 48 hours post-FACS of the sorted CD99 positive cells (A, Ai), CD99 negative cells (B, Bi), and “pass-through” control cells (C, Ci). Scale bar: 50 μm.

FIG. 29 shows the morphology of the cells in low (A, B) and high (Ai, Bi) magnification phase-contrast images acquired 5 days post-FACS of the sorted CD99 positive cells (A, Ai), and CD99 negative cells (B, Bi). Scale bar: 50 μm.

FIG. 30 shows representative immunofluorescence images of sorted CD99 positive (A), CD99 negative (B), and “pass-through” control (C) cells stained for Ki67 and DAPI at 48 hours post-FACS. Scale bar: 50 μm.

FIG. 31 shows representative immunofluorescence images of sorted CD99 positive (A), and CD99 negative (B) cells stained for Ki67 and DAPI at 12 days post-FACS. Scale bar: 50 μm.

FIG. 32 shows representative immunofluorescence images of sorted CD99 positive (A), CD99 negative (B), and “pass-through” control (C) cells stained for FOXA2 and DAPI at 48 hours post-FACS. Scale bar: 50 μm.

FIG. 33 shows representative immunofluorescence images of sorted CD99 positive (A), CD99 negative (B), and “pass-through” control (C) cells stained for TH and DAPI at 48 hours post-FACS. Scale bar: 50 μm.

FIG. 34 shows the percentage of DIV25 hPSC-derived midbrain cells expressing only CD47 (Q3) or CD99 (Q1) or both CD47 and CD99 (Q2).

FIG. 35 shows the quantification of Ki67+ cells by immunofluorescence after FACS. The graph displays the percentage of Ki67+ cells out of the total cells as identified by DAPI in the unsorted as well as CD47 negative and CD47 positive sorted samples.

FIG. 36 shows the quantification of SOX2+ cells by immunofluorescence after FACS. The graph displays the percentage of SOX2+ cells out of the total cells as identified by DAPI in the unsorted as well as CD47 negative and CD47 positive sorted samples.

FIG. 37 shows a bar graph of the results from intracellular flow cytometry analysis performed immediately after FACS. The graph displays the percentage of CD47+ cells out of the total viable cells in the unsorted sample as well as samples sorted based on CD47 (CD47− and CD47+) and samples sorted based on CD47 in combination with CD99 (CD47+CD99+ and CD47−CD99−).

FIG. 38 shows a bar graph of the results from intracellular flow cytometry analysis performed immediately after FACS. The graph displays the percentage of CD99+ cells out of the total viable cells in the unsorted sample as well as samples sorted based on CD47 (CD47− and CD47+) and samples sorted based on CD47 in combination with CD99 (CD47+CD99+ and CD47−CD99−).

FIG. 39 shows a bar graph of the results from intracellular flow cytometry analysis performed immediately after FACS. The graph displays the percentage of SOX2+ cells out of the total viable cells in the unsorted sample as well as samples sorted based on CD47 (CD47− and CD47+) and samples sorted based on CD47 in combination with CD99 (CD47+CD99+ and CD47−CD99−).

FIG. 40 shows a bar graph of the results from intracellular flow cytometry analysis performed immediately after FACS. The graph displays the percentage of Ki67+ cells out of the total viable cells in the unsorted sample as well as samples sorted based on CD47 (CD47− and CD47+) and samples sorted based on CD47 in combination with CD99 (CD47+CD99+ and CD47−CD99−).

FIG. 41 shows a bar graph of the results from intracellular flow cytometry analysis performed immediately after FACS. The graph displays the percentage of INA+ cells out of the total viable cells in the unsorted sample as well as samples sorted based on CD47 (CD47− and CD47+) and samples sorted based on CD47 in combination with CD99 (CD47+CD99+ and CD47−CD99−).

FIG. 42 shows the morphology of the cells in phase-contrast images acquired 48 hours post-MACS of the sorted CD47 negative cells (A) and unsorted control cells (B).

FIG. 43 shows post-sort purity immediately after MACS. The purity of the CD47 positive (A) and CD47 negative (B) cells was assessed by flow cytometry analysis.

FIG. 44 shows a bar graph summarizing the flow cytometry results obtained by intracellular flow cytometry immediately after MACS of unsorted cells (black bars) and CD47 negative cells (white bars).

FIG. 45 shows histograms displaying the percentage of DIV35 hPSC-derived forebrain cells expressing CD99 (A) and CD47 (B) measured by flow cytometry.

FIG. 46 shows the percentage of co-expression of SOX2 with Ki67 (A) and INA (B) in DIV35 forebrain cells as measured by flow cytometry.

FIG. 47 shows the percentage of co-expression of SOX2 with CD47 (A) and CD99 (B) in DIV35 forebrain cells as measured by flow cytometry.

FIG. 48 shows the percentage of co-expression of Ki67 with CD47 (A) and CD99 (B) in DIV35 forebrain cells as measured by flow cytometry.

FIG. 49 shows histograms displaying the percentage of DIV22 hPSC-derived hindbrain/spinal cord cells expressing CD99 (A) and CD47 (B) measured by flow cytometry.

FIG. 50 shows the percentage of co-expression of SOX2 with Ki67 (A) and INA (B) in DIV22 hindbrain/spinal cord cells as measured by flow cytometry.

FIG. 51 shows the percentage of co-expression of SOX2 with CD47 (A) and CD99 (B) in DIV22 hindbrain/spinal cord cells as measured by flow cytometry.

FIG. 52 shows the percentage of co-expression of Ki67 with CD47 (A) and CD99 (B) in DIV22 hindbrain/spinal cord cells as measured by flow cytometry.

FIG. 53 shows representative immunofluorescence images of unsorted control (A) and sorted CD47 negative (B) cells stained for TH and DAPI at 10 days post-MACS.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed.

Hereinafter, the methods according to the present invention are described in more detail by non-limiting embodiments and examples.

### Definitions

### General Definitions

As used herein, “a” or “an” or “the” can mean one or more than one. Unless otherwise indicated in the specification, terms presented in singular form also include the plural situation.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

### Stem Cells

By “stem cell” is to be understood an undifferentiated cell having differentiation potency and proliferative capacity (particularly self-renewal competence) but maintaining differentiation potency. The stem cell includes categories such as pluripotent stem cell, multipotent stem cell, unipotent stem cell and the like according to their differentiation potentiality.

As used herein, the term “pluripotent stem cell” (PSC) refers to a stem cell capable of being cultured in vitro and having a potency to differentiate into any cell lineage belonging to the three germ layers (ectoderm, mesoderm, endoderm).

A pluripotent stem cell can be induced or isolated from a fertilized egg, somatic nuclear transfer embryo, germ stem cell, stem cell in a tissue, somatic cell and the like. Examples of the pluripotent stem cell (PSC) include embryonic stem cell (ESC), embryonic germ cell (EG cell), induced pluripotent stem cell (iPSC) and the like.

As used herein, the term “induced pluripotent stem cell” (also known as iPS cells or iPSCs) means a type of pluripotent stem cell that can be generated directly from cells that are not PSCs and have a nucleus. By the introduction of products of specific sets of pluripotency-associated genes non-pluripotent cells can be converted into pluripotent stem cells.

As used herein, the term “embryonic stem cell” means a pluripotent stem cell derived from the inner cell mass of a blastocyst. Pluripotent embryonic stem cells may also be derived from parthenotes as described in e.g. WO 2003/046141. Additionally, embryonic stem cells can be produced from a single blastomere or by culturing an inner cell mass obtained without the destruction of the embryo. Embryonic stem cells are available from given organizations and are also commercially available. Preferably, the methods and products of the present invention are based on human PSCs, i.e. stem cells derived from either human induced pluripotent stem cells or human embryonic stem cells, including parthenotes.

As used herein, the term “multipotent stem cell” means a stem cell having a potency to differentiate into plural types of tissues or cells, though not all kinds and is typically restricted to one germ layer. A neural stem cell is an example of a multipotent stem cell restricted to the neural lineage.

As used herein, the term “unipotent stem cell” means a stem cell having a potency to differentiate into only one particular cell type.

As used herein, the term “in vitro” means that the cells are provided and maintained outside of the human or animal body, such as in a vessel like a flask, multiwell or petri dish. It follows that the cells are cultured in a cell culturing medium.

As used herein, the term “non-native” means that the cells although derived from pluripotent stem cells, which may have human origin, is an artificial construct, that does not exist in nature. In general, it is an object within the field of stem cell therapy to provide cells, which resemble the cells of the human body as much as possible. However, it may never become possible to mimic the development which the pluripotent stem cells undergo during the embryonic and fetal stage to such an extent that the mature cells are indistinguishable from native cells of the human body. Inherently, in an embodiment of the present invention, the cells are artificial.

As used herein, the term “artificial” in reference to cells may comprise material naturally occurring in nature but modified to a construct not naturally occurring. This includes human stem cells, which are differentiated into non-naturally occurring cells mimicking the cells of the human body.

When referring to a certain percentage of a cell population, such as X % of the cell population express a certain marker, is meant the percentage of cells in the cell population, i.e. X % of cells in the cell population.

### Protocol

Throughout this application the terms “method” and “protocol” when referring to processes for culturing or differentiating cells may be used interchangeably.

As used herein, the term “day” and similarly day in vitro (DIV) in reference to the protocols refers to a specific time for carrying out certain steps during the differentiation procedure.

In general and unless otherwise stated “day 0” refers to the initiation of the protocol, this is by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound or compounds that affects the undifferentiated stem cells in such a way that a differentiation process is initiated.

When referring to “the cells” in a method is meant all cells of the cell population, regardless of cell type.

When referring to “day X”, such as day 1, day 2 etc., it is relative to the initiation of the protocol at day 0. One of ordinary skill in the art will recognize that unless otherwise specified the exact time of the day for carrying out the step may vary. Accordingly, “day X” is meant to encompass a time span such as of +/−10 hours, +/−8 hours, +/−6 hours, +/−4 hours, +/−2 hours, or +/−1 hours.

### Culturing Stem Cells

As used herein, the term “culturing” refers to a continuous procedure, which is employed throughout the method in order to maintain the viability of the cells at their various stages. After the cells of interest have been isolated from, for example but not limited to, living tissue or embryo, they are subsequently maintained under carefully controlled conditions. These conditions vary for each cell type, but generally consist of a suitable vessel with a substrate and/or medium that supplies the essential nutrients (amino acids, carbohydrates, vitamins, minerals), growth factors, hormones, and gases (CO₂, O₂), and regulates the physio-chemical environment (pH buffer, osmotic pressure, temperature).

As used herein, the term “cell culture medium” refers to a liquid or gel designed to support the growth of cells. Cell culture media generally comprise an appropriate source of energy and compounds which regulate the cell cycle.

As used herein, the term “incubator” refers to any suitable incubator that may support a cell culture. Non-limiting examples include culture dish, petri dish and plate (microtiter plate, microplate, deep well plate etc. of 6 well, 24 well, 48 well, 96 well, 384 well, 9600 well and the like), flask, chamber slide, tube, Cell Factory systems, roller bottle, spinner flask, hollow fiber, microcarrier, or bead.

As used herein, the term “providing stem cells” when referred to in a protocol means obtaining a batch of cells by methods such as described above and optionally transferring the cells into a different environment such as by seeding onto a new substrate. One of ordinary skill in the art will readily recognize that stem cells are fragile to such transfer and the procedure requires diligence and that maintaining the stem cells in the origin cell culture medium may facilitate a more sustainable transfer of the cells before replacing a cell culture medium with another cell culture medium more suitable for a further differentiation process.

### Differentiating Stem Cells

As used herein, the term “expressing” in relation to a gene or protein refers to the presence of an RNA molecule, which can be detected using assays such as reverse transcription quantitative polymerase chain reaction (RT-qPCR), RNA sequencing and the like, and/or a protein, which can be detected for example using antibody-based assays such as flow cytometry, immunocytochemistry/immunofluorescence, and the like. Depending on the sensitivity and specificity of the assay, a gene or protein may be considered expressed when a minimum of one molecule is detected such as in RNA sequencing, or the limit of detection above background/noise levels may be defined in relation to control samples such as in flow cytometry. The expression of markers in a cell or cell population may be determined by the method as described in Example 9.

As used herein, the term “marker” refers to a naturally occurring identifiable expression made by a cell, which can be correlated with certain properties of the cell. In a preferred embodiment the marker is a genetic or proteomic expression, which can be detected and correlated with the identity of the cell. The markers may be referred to by gene. This can readily be translated into the expression of the corresponding mRNA and proteins.

As used herein, the term “negative” or “−” when used in reference to any marker such as a surface protein or transcription factor disclosed herein refers to the marker not being expressed in a cell or a population of cells, while the term “weak” or “low” refers to the marker being expressed at a reduced level in a cell as compared to the mean expression of the marker in a population of cells or as compared to a reference sample.

As used herein, the term “positive” or “+” when used in reference to any marker such as a surface protein or transcription factor disclosed herein refers to the marker being expressed in a cell or a population of cells, while the term “high” or “strong” refers to the marker being expressed at an increased level in a cell as compared to the mean expression of the marker in a population of cells or as compared to a reference sample.

As used herein, the term “differentiation” refers broadly to the process wherein cells progress from an undifferentiated state or a state different from the intended differentiated state to a specific differentiated state, e.g. from an immature state to a less immature state or from an immature state to a mature state, which may occur continuously as the method is performed. The term “differentiation” in respect to pluripotent stem cells refers to the process wherein cells progress from an undifferentiated state to a specific differentiated state, i.e. from an immature state to a less immature state or to a terminal state. Changes in cell interaction and maturation occur as cells lose markers of undifferentiated cells or gain markers of differentiated cells. Loss or gain of a single marker can indicate that a cell has “fully differentiated” or “terminally differentiated”. “Terminally differentiated” cells are the final stage of a developmental lineage and cannot further differentiate.

As used herein, by the term “contacting” in reference to culturing or differentiating cells is meant exposing the cells to e.g. a specific compound by placing the specific compound in a location that will allow it to touch the cell in order to produce “contacted” cells. The contacting may be accomplished using any suitable means. A non-limiting example of contacting is by adding the compound to a cell culture medium of the cells. The contacting of the cells is assumed to occur as long as the cells and specific compound are in proximity, e.g. the compound is present in a suitable concentration in the cell culture medium.

As used herein, the term “inhibitor” refers to a compound that reduces or suppresses or down-regulates a process, such as a signaling pathway which can promote cell differentiation.

As used herein, the term “activator” refers to a compound that induces or stimulates or up-regulates a process, such as a signaling pathway which can promote cell differentiation.

As used herein, when describing the steps of a protocol the cells may be referred to as “cells”, “differentiating cells” or in some cases PSCs or “PSC-derived cells”. A skilled person will recognize that during a protocol for differentiating PSCs into specialized cells, the cells at some point lose their pluripotency. Accordingly, when referring the “cells” or “PSCs” in a step of e.g. contacting the cells with a compound is meant the cells which initially were pluripotent stem cells.

### Stem Cell Products

As used herein, the term “differentiated cells” refers to cells such as pluripotent stem cells which have progressed from an undifferentiated state to a less immature state. Differentiated cells may be e.g. less immature specialized cell such as progenitor cells or matured fully into a specialized/terminal cell type.

As used herein, the term “cell population” refers to a plurality of cells in the same culture. The cell population may be e.g. a mixture of cells of different types or cells at various developmental stages such as cells at various maturity stages towards the same or similar specialized feature or it may be a more homogeneous composition of cells with common markers. As used herein, reference to a proportion of a cell population or cells, such as “X % of a cell population” and “X % of the cells”, may be used interchangeably.

As used herein, the term “neural cell population” refers to a cell population comprising neural cells.

As used herein, the term “genetically modified” in reference to a cell refers to a cell which has been subjected to gene editing and can no longer be considered a naturally occurring cell. In case of genetically modified stem cells the traits resulting from the gene editing persist even as the stem cell is further differentiated into a specialized cell, thus rendering the specialized cell genetically modified and artificial, i.e. non-naturally occurring. An example of genetically modified stem cells are HLA-deficient stem cells, which are also referred to as universal donor cells and intended to overcome the problem of graft rejection. A method for obtaining HLA-deficient stem cells is disclosed in WO/2020/260563.

### Neuroectodermal Cells

As used herein, the term “neural” refers to the nervous system.

As used herein, the term “neural cell” refers to a non-native cell, where the native counterpart naturally forms part of the ectoderm germ layer, more specifically the neuroectoderm and is meant to encompass cells at any stage of development within this germ layer, such as neural stem cells all the way through to neurons and other terminally differentiated cell types (e.g. glial cells), i.e. cell stages such as neural stem cell stage and neuroblast stage. Accordingly, neurons and precursors thereof are considered specific types of neural cells.

As used herein, the terms “neuron” and “nerve cell” may be used interchangeably referring to neural cells which are post-mitotic and have terminally differentiated into a specialized cell. Neuron identity is characterized by the expression of one or more of the markers INA, STMN2, TUBB3, MAP2, and RBFOX3.

As used herein, the term “neuroblast cell” refers to an intermediate precursor cell, which is typically multipotent or only unipotent and can self-renew only to a limited extent. A neuroblast cell finally gives rise to terminally differentiated cell types such as neurons or astrocytes. The terms “neuroblast cell” and “intermediate precursor cell” and “intermediate progenitor cell” may be used interchangeably. Intermediate precursor cell identity is characterized by the expression of one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2 and NHLH1.

As used herein, the terms “neuron progenitor”, “precursor of a neuron” and “neuron precursor” may be used interchangeably and refer to a neural cell with the potential or propensity to further specialize into a neuron. The terms “neuron precursor” and “non-native neuron precursor” may be used interchangeably.

The neural cells according to the present invention may have a specific regional identity, such as cells specific to the forebrain, midbrain, hindbrain, spinal cord etc.

As used herein, the term “forebrain” refers to the rostral region of the neural tube and CNS that gives rise to structures including the cerebral cortex and the striatum.

As used herein, the term “midbrain” refers to the medial region of the neural tube and CNS (on the rostro-caudal axis) that gives rise to structures including the substantia nigra.

As used herein, the terms “hindbrain” and “spinal cord” refer to the caudal regions of the neural tube that are caudal to the isthmus organizer.

As used herein, the term “dopaminergic (DA) cell” or “dopaminergic neuron” or “dopamine neuron” refers to a cell that is capable of synthesizing the neurotransmitter dopamine.

Method for Enriching a Cell Population in Neurons and/or Precursors Thereof

An aspect of the present invention relates to a method for providing a cell population enriched in neurons and/or precursors thereof, comprising the steps of obtaining a cell population comprising neuron precursors, selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

Another aspect of the present invention relates to a method for enriching a cell population in neurons and/or precursors thereof, comprising the steps of obtaining a cell population comprising neuron precursors, selecting cells positive for one or more positive markers, and/or excluding cells positive for one or more negative markers.

As used herein, the term “enriching in” in reference to a cell population means increasing the proportion of a desired type of cells in the cell population. Specifically, as used herein, the term “enriching a cell population in neurons and/or precursors thereof” means increasing the proportion of neurons and/or precursors thereof in the cell population. It follows that a cell population “enriched in” means that the cell population has an increased proportion of a desired type of cells as compared to a reference cell population which has not been subject to a method of enriching in said desired type of cells. In a preferred embodiment, the cell population is enriched by isolating the desired type of cells. As used herein, the term “isolating” in reference to a cell population means separating the desired type of cells from unwanted cells, either by removing the unwanted cells from the cell population or by recovering the desired type of cells from the cell population. Accordingly, the method for enriching the cell population in neurons and/or precursors thereof may alternatively be worded as a method for isolating neurons and/or precursors thereof from a population of cells, and embodiments described herein apply equally to both.

Therefore, the present invention relates to a method for isolating neurons and/or precursors thereof from a cell population, comprising the steps of obtaining a cell population comprising neural stem cells, and selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

In an embodiment, the neural stem cells are of the forebrain, midbrain, hindbrain, or spinal cord region. In an embodiment, the neural stem cells are ventral midbrain neural stem cells.

As used herein, the term “obtaining” in reference to a cell population comprising neural stem cells and/or neuron precursors means acquiring a batch of cells obtainable according to any method for inducing differentiation of cells, such as PSCs, wherein the cells are differentiated into neural cells comprising neural stem cells and/or neuron precursors.

As used herein, the term “selecting” in reference to a cell population comprising cells expressing a certain marker means carrying out a step intended to result in a cell population with an increased fraction of the cells expressing the marker as compared to the fraction of cells not expressing the marker, as compared to the initial cell population prior to carrying out the step. Any suitable method may be used for selecting cells expressing a certain marker, such as isolating the cells expressing the marker or separating the cells.

As used herein, the term “excluding” in reference to cells expressing a certain marker means carrying out a step intended to result in a cell population with a decreased fraction of the cells expressing the marker as compared to the fraction of cells not expressing the marker, as compared to the initial cell population prior to carrying out the step. Any suitable method may be used for excluding cells expressing a certain marker, such as depleting the cells expressing the marker or separating the cells. A non-limiting example of a method for selecting and/or excluding cells expressing a certain marker is antibody-mediated sorting, such as fluorescent-activated cell sorting (FACS), magnetic-activated cell sorting (MACS), or immunopanning. It follows that in a preferred embodiment, the positive and negative markers are surface markers.

As used herein, the term “positive markers” refers to markers, which can be used to select cells and includes the markers PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85.

As used herein, the term “negative markers” refers to markers, which can be used to exclude cells and includes the markers SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

In an embodiment, the method comprises obtaining a cell population comprising neuron precursors. A cell population comprising neuron precursors may be obtained by initially obtaining a cell population comprising neural stem cells expressing the marker SOX2, wherein the cell population is further cultured until the expression of SOX2 in the cell population decreases, thereby indicating that the neural stem cells have further differentiated into intermediate precursor cells, some of which being neuron precursors. At this stage the surface markers according to the method are suitable for selection and/or exclusion of cells to isolate the cells having neuronal fate. Accordingly, in an embodiment, the cell population comprising neuron precursors is obtained by the steps of obtaining a cell population comprising neural stem cells expressing the marker SOX2, and culturing the cell population comprising neural stem cells until the expression of SOX2 in the cell population comprising neural stem cells has decreased. In an embodiment, at least 80%, such as at least 85%, 90%, or 95%, preferably at least 95%, of the obtained cell population comprising neural stem cells express SOX2. In an embodiment, the cell population comprising neural stem cells is cultured until the expression of SOX2 in the cell population has decreased by at least 5%, such as by 10%, 15%, 20%, 25%, 30%, 35%, or 40%, preferably by at least 20%. In an embodiment, at least 90% of the obtained cell population comprising neural stem cells express SOX2, and the cells are further cultured until at least less than 90% express the marker SOX2, preferably until less than 80% express the marker SOX2, more preferably until less than 70% express the marker SOX2.

In an embodiment, the selecting and/or excluding in step c) is carried out within 5 days, such as within 4 days, 3 days, or 2 days, after less than 80% of the cell population comprising neural stem cells express the marker SOX2.

In another embodiment, the timing for selecting and/or excluding cells is determined based on the expression of CD99 instead of SOX2. Accordingly, in an embodiment, at least 80% of the cell population comprising neural stem cells obtained in step a) express CD99. In an embodiment, the cell population is further cultured in step b) at least until the expression of CD99 in the cell population comprising neural stem cells has decreased by 5%, such as by 10%, 15%, 20%, 25%, 30%, 35%, or 40%. In an embodiment, at least 90% of the cell population comprising neural stem cells obtained in step a) express CD99, and wherein the cells are further cultured in step b) until at least less than 90% express the marker CD99, preferably until less than 80% express the marker CD99, more preferably until less than 70% express the marker CD99. And in an embodiment, selecting and/or excluding in step c) is carried out within 5 days, such as within 4 days, 3 days, or 2 days, after less than 80% of the cell population comprising neural stem cells express the marker CD99.

In a preferred embodiment, the cell population comprises intermediate precursor cells and/or neurons when selecting and/or excluding the cells based on the positive and negative markers. In an embodiment, at least 5%, such as at least 10%, 15%, 20%, 25%, 30%, 35%, or 40%, of the cell population express intermediate precursor identity or neuron identity when carrying out the step of selecting and/or excluding the cells. In an embodiment, the cell population is cultured until the expression of one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1 is at least 5%, such as at least 10%, at least 15%, or at least 20%. In an embodiment, the cell population is cultured until the expression of one or more of the markers INA, STMN2, TUBB3, MAP2, and RBFOX3 is at least 5%, such as at least 10%, at least 15%, or at least 20%.

In an embodiment, at least 5%, at least 10%, at least 15%, at least 20%, at least 25%, at least 30%, at least 35%, or at least 40% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, NHLH1, INA, STMN2, TUBB3, MAP2, and RBFOX3 when carrying out the steps of selecting and/or excluding the cells.

In an embodiment, at the time of selecting and/or excluding cells, 10-90%, such as 10-80%, 10-70%, 10-60%, or 10-50%, of the cell population are positive for SOX2, 5-50%, such as 5-40%, 5-30%, 5-20%, of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1, and 5-20%, such as 5-15%, or 5-10%, of the cell population are positive for one or more of the markers INA, TUBB3, STMN2, MAP2, and RBFOX3.

In an embodiment, wherein the neural cells are of the ventral midbrain identity, at least 5%, such as at least 10%, at least 15%, at least 20%, at least 25%, at least 30%, at least 35%, or at least 40% of the cell population are positive for one or both of the markers ASCL1 and INA when carrying out the steps of selecting and/or excluding the cells.

In an embodiment, the cells positive for the marker CD47 are excluded. In an embodiment, the cells positive for the marker CD99 are excluded.

In an embodiment, the method comprises the further step of maturing neurons and/or precursors thereof following the steps of selection and/or exclusion.

In an embodiment, the method comprises the further step of recovering the neural cell population enriched in neurons and/or precursors thereof following the steps of selection and/or exclusion and optionally following a further step of maturing neurons and/or precursors thereof.

In an embodiment, the cells recovered are negative for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

In an embodiment, the cell population is a neural cell population. In an embodiment, at least 50%, more preferably at least 55%, more preferably at least 60%, more preferably at least 65%, more preferably at least 70%, more preferably at least 75%, more preferably at least 80%, more preferably at least 85%, or more preferably at least 90%, more preferably at least 95%, even more preferably at least 99% of the cell population are neural cells.

In an embodiment, the enriched cell population has an increased propensity for developing into neurons. As used herein, the term “propensity” in reference to cell specialization refers to the likelihood of a cell to develop into a certain cell type. Accordingly, increasing the propensity of a cell population to develop into a certain cell type means increasing the proportion of cells which are likely to develop into that cell type.

In an embodiment, at least 50%, at least 60%, at least 70%, at least 80%, at least 90%, or at least 95% of the neurons and/or precursors thereof are of the ventral midbrain, ventral spinal cord, dorsal forebrain, or ventral forebrain region.

In a particular embodiment, at least 50%, at least 60%, at least 70%, at least 80%, at least 90%, at least 95%, or at least 99% of the neurons and/or precursors thereof are of the ventral midbrain region.

In a certain embodiment, the cell population of the ventral midbrain region, and the cells positive for the marker CD47 are excluded at a time where at least 5%, such as 10%, 15%, 20%, 25%, 30%, 35%, 40%, 45%, 50%, 55%, 60%, 65%, or 70%, of the cell population express one of the markers ASCL1 and INA. In a further embodiment, the cells positive for any of the markers CD47 and CD99 are excluded.

In another embodiment, the cell population of the ventral spinal cord, dorsal forebrain or ventral forebrain, and the cells positive for the marker CD99 are excluded.

In a certain embodiment, the selection and/or exclusion of the cells is carried out by antibody-mediated cell sorting. In a further embodiment, the selection and/or exclusion of the cells is carried out using fluorescent-activated cell sorting (FACS), magnetic-activated cell sorting (MACS), or immunopanning or as compared to a reference sample, preferably the selection and/or exclusion is carried out using MACS.

In an embodiment, the neural cell population is an in vitro cell culture. In an embodiment, the cells of the neural cell population are non-native.

A person skilled in the art will recognize that expression of certain markers indicative of the cell stage may be expressed internally in the cell and is to be analyzed by e.g. single cell RNA-seq, whereas other markers are expressed on the cell surface and antibody-mediated analysis may be used, such as FACS. Timing of selection and/or exclusion of cells based on the surface markers may be established by analyzing samples for expression of e.g. SOX2. Typically, during design of a protocol for obtaining e.g. neural stem cells, the cell expression during the protocol is analyzed so that timing of selection and/or exclusion in the protocol can be determined without further need to monitor the expression of the markers during a production set up.

### Method for Obtaining Neural Cells Comprising Neuron Precursors

In an embodiment, the cell population comprising neuron precursors is derived from PSCs, preferably hPSCs. In a certain embodiment, the hPSCs are human embryonic stem cells or human induced pluripotent stem cells.

In an embodiment of the method, obtaining the cell population comprising neuron precursors comprises the step of allowing PSCs to differentiate into neural cells comprising neuron precursors. In a certain embodiment, the PSCs are contacted with at least one inhibitor of the SMAD signaling pathway.

As used herein, the term “SMAD signaling pathway” refers to the Small Mothers Against Decapentaplegic (SMAD) protein signaling pathway. In an embodiment, the at least one inhibitor of the SMAD signaling pathway is selected from Noggin, LY364947, SB431542, and LDN-193189. In an embodiment, the PSCs are contacted with two or more inhibitors of the SMAD signaling pathway. A person skilled in the art will recognize that contacting PSCs with one or more inhibitors of the SMAD signaling pathway is a robust technique for differentiating cells into the neural lineage.

In an embodiment, the PSCs are contacted with one or more inhibitors of the SMAD signaling pathway at day 0. In a further embodiment, the PSCs are contacted with the one or more inhibitors of the SMAD signaling pathway for at least 5 days, such as for at least 7 days, for at least 8 days, for at least 9 days, or for at least 10 days.

In an embodiment of the method, obtaining the cell population comprising neuron precursors comprises the step of allowing PSCs to differentiate into neural cells of the forebrain, midbrain, hindbrain, or spinal cord region. In an embodiment, wherein the cells are differentiated into the midbrain, hindbrain or spinal cord regions, the cells are contacted with at least one activator of the WNT pathway. In an embodiment, the cells are contacted with the at least one activator of the WNT signaling pathway within day 0 to day 5, preferably at day 0. In an embodiment, the cells are contacted with the at least one activator of the WNT signaling pathway for at least 5 days, such as at least 6 days, at least 7 days, at least 8 days, at least 9 days. In an embodiment, the cells are contacted with at least one activator of the WNT signaling pathway simultaneously with contacting the cells with the one or more inhibitors of the SMAD signaling pathway.

As used herein, the term “WNT pathway” refers to the WNT signaling pathway. In an embodiment, at least one activator of the WNT pathway is a GSK3 inhibitor, such as CHIR99021. Accordingly, in an embodiment, the PSCs are contacted with CHIR99021.

In an embodiment of the method, obtaining the cell population comprising neuron precursors comprises the step of allowing PSCs to differentiate into neural cells of the ventral region of the neural tube. In such an embodiment, the cells may be contacted with an activator of the SHH signaling pathway. As used herein, the term “SHH” refers to Sonic hedgehog. In an embodiment, the cells are contacted with a compound selected from Sonic Hedgehog C24II, Purmorphamine, and SAG (smoothened agonist). In an embodiment, the cells are contacted with an activator of the SHH signaling pathway from at least day 7, such as at least day 6, at least day 5, at least day 4, at least day 3, at least day 2, at least day 1, or day 0. In a preferred embodiment, the cells are contacted with the activator of the SHH signaling pathway simultaneously with contacting the cells with the one or more inhibitors of the SMAD signaling pathway.

In an embodiment of the method, obtaining the cell population comprising neuron precursors comprises the step of allowing PSCs to differentiate into neural cells of the midbrain or hindbrain region. In such an embodiment, the cells may be contacted with FGF8, preferably FGF8b. As used herein, the term “FGF” refers to fibroblast growth factor. In an embodiment, the cells are contacted with FGF8 at between day 5 and day 15, preferably between day 8 and day 10, such as at day 9. In an embodiment, the cells are contacted with FGF8 for at least 2 days, such as at least 5 days or 6 days. In an embodiment, the cells are contacted with FGF8 when ending exposure of the cells to the one or more inhibitors of the SMAD signaling pathway.

In an embodiment of the method, obtaining the cell population comprising neuron precursors comprises the step of allowing PSCs to differentiate into neural cells of the ventral midbrain region.

In an embodiment for differentiating neural cells into the ventral midbrain region, the cells are contacted with ascorbic acid, such as L-ascorbic acid. In an embodiment, the cells are contacted with ascorbic acid after ending exposure of the cells to the one or more inhibitors of the SMAD signaling pathway. In an embodiment, the cells are contacted with ascorbic acid from day 9, day 10, day 11, day 12, or day 13, preferably from day 11. In a preferred embodiment, the cells are contacted with ascorbic acid until the step of recovering the neural cells.

In an embodiment for differentiating neural cells into the ventral midbrain region, the cells are contacted with BDNF. As used herein, the term “BDNF” refers to brain-derived neurotrophic factor. In an embodiment, the cells are contacted with BDNF after ending exposure of the cells to the one or more inhibitors of the SMAD signaling pathway. In an embodiment, the cells are contacted with BDNF from day 9, day 10, day 11, day 12, or day 13, preferably from day 11. In a preferred embodiment, the cells are contacted with BDNF until the step of recovering the neural cells. In an embodiment, the cells are contacted with ascorbic acid and BDNF simultaneously.

In an embodiment for differentiating neural cells into the ventral midbrain region, the cells are contacted with GDNF. As used herein, the term “GDNF” refers to glial-derived neurotrophic factor. In an embodiment, the cells are contacted with GDNF after ending exposure of the cells to the one or more inhibitors of the SMAD signaling pathway. In an embodiment, the cells are contacted with GDNF from day 11, day 12, day 13, day 14, day 15, day 16, day 17, or day 18, preferably from day 16. In a preferred embodiment, the cells are contacted with GDNF until the step of recovering the neural cells.

In an embodiment for differentiating neural cells into the ventral midbrain region, the cells are contacted with DAPT or other inhibitors of the Notch pathway. As used herein, the term “DAPT” refers to (2S)—N-[(3,5-Difluorophenyl)acetyl]-L-alanyl-2-phenyl]glycine 1,1-dimethylethyl ester. In an embodiment, the cells are contacted with DAPT after ending exposure of the cells to the one or more inhibitors of the SMAD signaling pathway. In an embodiment, the cells are contacted with DAPT from day 11, day 12, day 13, day 14, day 15, day 16, day 17, or day 18, preferably from day 16. In a preferred embodiment, the cells are contacted with DAPT until the step of recovering the neural cells. In an embodiment, the cells are contacted with GDNF and DAPT at the same time.

In an embodiment, the PSCs are cultured and differentiated on a substrate comprising an extracellular matrix, such as Poly-L-Lysine, Poly-D-Lysine, Poly-L-Ornithine, laminin, fibronectin, iMatrix and/or collagen, and/or fragments thereof. In an embodiment, the substrate comprises laminin or fragments thereof, such as laminin-111, laminin-521, and/or laminin-511.

In an embodiment, the PSCs are allowed to differentiate into neural cells comprising neuron precursors for at least 10 days, such as at least 15 days, at least 18 days, at least 20 days, at least 22 days, at least 25 days, or at least 30 days, preferably at least 20 days, prior to the step of selecting and/or excluding the cells.

In an embodiment, the PSCs are allowed to differentiate into neural cells comprising neuron precursors for 15 days to 50 days, preferably 18 days to 40 days, more preferably 20 days to 40 days, more preferably 20 days to 30 days, prior to the step of selecting and/or excluding the cells. In an embodiment, the remaining neurons and/or precursors thereof are allowed to further differentiate/mature following the step of selecting and/or excluding of cells.

In an embodiment, the step of selecting and/or excluding cells is carried out between day 15 and day 40 from initiating the differentiation of the PSCs into neural cells comprising neuron precursors.

In an embodiment, the neural cells are differentiated into ventral midbrain neural cells and the step of selecting and/or excluding cells is carried out at from day 22 to day 25.

Cell Population Enriched in Neurons and/or Precursors Thereof

Another aspect of the present invention relates to a cell population of neurons and precursors thereof, wherein at least 50% of the cells are positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or less than 50% of the cells are positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

In an embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85.

In an embodiment, less than 50%, preferably less than 45%, more preferably less than 40%, more preferably less than 35%, more preferably less than 30%, more preferably less than 25%, more preferably less than 20%, more preferably less than 15%, more preferably less than 10%, more preferably less than 5%, or even more preferably less than 1% of the cells are positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.

In an embodiment, less than 50%, preferably less than 45%, more preferably less than 40%, more preferably less than 35%, more preferably less than 30%, more preferably less than 25%, more preferably less than 20%, more preferably less than 15%, more preferably less than 10%, more preferably less than 5%, or even more preferably less than 1% of the cells are positive for the marker CD47.

In an embodiment, less than 50%, preferably less than 45%, more preferably less than 40%, more preferably less than 35%, more preferably less than 30%, more preferably less than 25%, more preferably less than 20%, more preferably less than 15%, more preferably less than 10%, more preferably less than 5%, or even more preferably less than 1% of the cells are positive for the marker CD99.

In an embodiment, less than 50%, preferably less than 45%, more preferably less than 40%, more preferably less than 35%, more preferably less than 30%, more preferably less than 25%, more preferably less than 20%, more preferably less than 15%, more preferably less than 10%, more preferably less than 5%, or even more preferably less than 1% of the cells are positive for the markers CD47 and CD99.

In an embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are selected from ventral midbrain neurons and/or precursors thereof, ventral spinal cord neurons and/or precursors thereof, dorsal forebrain neurons and/or precursors thereof, and ventral forebrain neurons and/or precursors thereof.

In an embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are positive for the marker FOXA2.

In an embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are ventral midbrain neurons and/or precursors thereof.

In a certain embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are neuroblast cells and/or neurons.

In an embodiment, at least 50%, preferably at least 55%, more preferably at least 60%, more preferably at least 65%, more preferably at least 70%, more preferably at least 75%, more preferably at least 80%, more preferably at least 85%, more preferably at least 90%, more preferably at least 95%, or even more preferably at least 99% of the cells are positive for one or both of the markers ASCL1 and INA.

In an embodiment, the cell population has an increased propensity for developing into neurons.

In an embodiment, the cell population is in vitro.

In an embodiment, the cell population is a neural cell population.

In an embodiment, the cell population is enriched in neurons and/or precursors thereof.

In an embodiment, the cells of the cell population are in vitro differentiated cells.

In an embodiment, the cells of the cell population are non-native cells.

In an embodiment, the cells of the cell population are derived from hPSCs.

In an embodiment, the cells of the cell population are genetically modified. In a further embodiment, the genetically modified cells are HLA-deficient.

In an embodiment, the cell population comprises at least 1,000 cells, such as at least 100,000 cells, at least 1,000,000 cells, or at least 10,000,000 cells.

In an embodiment, the cell population is obtained by any of the methods as described above.

### Pharmaceutical Composition and Use as a Medicament

An aspect of the present invention relates to a composition comprising an enriched cell population as described herein. The composition may comprise any suitable additives which are pharmacologically acceptable, such as cryoprotectants and/or biomaterials, such as extracellular matrices.

Another aspect relates to a composition or cell population according to the present invention for use as a medicament. Accordingly, in an embodiment, the composition or cell population is for the prevention or treatment of a condition requiring the administration of neurons and/or precursors thereof.

In an embodiment, the composition or cell population is for use in the treatment of a neurological condition, such as Parkinson's disease, stroke, traumatic brain injury, spinal cord injury, Huntington's disease, dementia, epilepsy, blindness, Alzheimer's disease, and other neurological conditions wherein neurons are lost or dysfunctional.

In a specific embodiment, the cell population comprises ventral midbrain neurons and/or precursors thereof for the treatment of Parkinson's disease.

In a specific embodiment, the cell population comprises cortical neurons and/or precursors thereof for the treatment of Stroke injuries.

In another aspect is provided a method of treatment or prevention of a neurological condition comprising the administration to a patient of an effective amount of a cell population or composition according to the present invention.

### Particular Embodiments

The aspects of the present invention are now further described by the following non-limiting embodiments:
1. A method for isolating neurons and/or precursors thereof from a cell population, comprising the steps of:
   a) obtaining a cell population comprising neural stem cells expressing SOX2,
   b) further culturing the cell population comprising neural stem cells,
   c) selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.
2. The method according to the preceding embodiment, wherein at least 80% of the cell population comprising neural stem cells obtained in step a) express SOX2.
3. The method according to any one of the preceding embodiments, wherein the cell population is further cultured in step b) at least until the expression of SOX2 in the cell population comprising neural stem cells has decreased by 5%, such as by 10%, 15%, 20%, 25%, 30%, 35%, or 40%.
4. The method according to any one of the preceding embodiments, wherein at least 90% of the cell population comprising neural stem cells obtained in step a) express SOX2, and wherein the cell population is further cultured in step b) until at least less than 90% express the marker SOX2, preferably until less than 80% express the marker SOX2, more preferably until less than 70% express the marker SOX2.
5. The method according to any one of the preceding embodiments, wherein selecting and/or excluding in step c) is carried out within 5 days, such as within 4 days, 3 days, or 2 days, after less than 80% of the cell population comprising neural stem cells express the marker SOX2.
6. The method according to any one of the preceding embodiments, wherein at least 80% of the cell population comprising neural stem cells obtained in step a) express CD99.
7. The method according to any one of the preceding embodiments, wherein the cell population is further cultured in step b) at least until the expression of CD99 in the cell population comprising neural stem cells has decreased by 5%, such as by 10%, 15%, 20%, 25%, 30%, 35%, or 40%.
8. The method according to the preceding embodiment, wherein at least 90% of the cell population comprising neural stem cells obtained in step a) express CD99, and wherein the cell population is further cultured in step b) until at least less than 90% express the marker CD99, preferably until less than 80% express the marker CD99, more preferably until less than 70% express the marker CD99.
9. The method according to any one of the preceding embodiments, wherein selecting and/or excluding in step c) is carried out within 5 days, such as within 4 days, 3 days, or 2 days, after less than 80% of the cell population comprising neural stem cells express the marker CD99.
10. The method according to any one of the preceding embodiments, wherein the cell population is further cultured in step b) until the expression of one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1 is at least 5%, such as 10%, 15%, or 20%,
11. The method according to any one of the preceding embodiments, wherein the cell population is further cultured in step b) until the expression of one or more of the markers INA, STMN2, TUBB3, MAP2, and RBFOX3 is at least 5%, such as 10%, 15%, or 20%.
12. The method according to any one of the preceding embodiments, wherein at the time of selecting and/or excluding cells in step c), 10-90% of the cell population are positive for SOX2, 5-50% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1, and 5-20% of the cell population are positive for one or more of the markers INA, TUBB3, STMN2, MAP2, and RBFOX3.
13. The method according to any one of the preceding embodiments, wherein the cell population is a neural cell population.
14. The method according to any one of the preceding embodiments, wherein the cell population comprising neural stem cells in step a) is obtained by neural induction of PSCs for at least 14 days.
15. The method according to any one of the preceding embodiments, wherein the neural stem cells are of the forebrain, midbrain, hindbrain, or spinal cord region.
16. The method according to any one of the preceding embodiments, wherein the neural stem cells are ventral midbrain neural stem cells.
17. A method for providing a cell population enriched in neurons and/or precursors thereof, comprising the steps of:
   obtaining a cell population comprising neuron precursors,
   selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or
   excluding cells positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.
18. The method according to embodiment 17, wherein the cell population comprising neuron precursors is obtained by the steps of obtaining a cell population comprising neural stem cells expressing the marker SOX2, and culturing the cell population comprising neural stem cells until the expression of the marker SOX2 in the cell population comprising neural stem cells decreases.
19. The method according to embodiment 18, wherein at least 80% of the cell population comprising neural stem cells obtained express the marker SOX2.
20. The method according to embodiment 19, wherein the cell population is further cultured at least until the expression of SOX2 in the cell population comprising neural stem cells has decreased by 5%, such as by 10%, 15%, 20%, 25%, 30%, 35%, or 40%.
21. The method according to any one of embodiments 18 and 19, wherein at least 80% of the cell population comprising neural stem cells is positive for SOX2, and wherein the cell population comprising neural stem cells is cultured until the expression of SOX2 is less than 80%.
22. The method according to any one of the preceding embodiments, wherein cells positive for the marker CD47 are excluded.
23. The method according to any one of the preceding embodiments, wherein cells positive for the marker CD99 are excluded.
24. The method according to any one of the preceding embodiments, where cells positive for any of the markers CD99 and CD47 are excluded.
25. The method according to any one of the preceding embodiments, wherein at least 80% of the cell population are neural cells.
26. The method according to any one of the preceding embodiments, wherein at least 5% of the cell population express intermediate precursor identity or neuron identity when selecting and/or excluding the cells.
27. The method according to any one of the preceding embodiments, wherein at least 5% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1 when selecting and/or excluding cells.
28. The method according to any one of the preceding embodiments, wherein at least 5% of the cell population are positive for one or more of the markers INA, TUBB3, STMN2, MAP2, and RBFOX3 when selecting and/or excluding cells.
29. The method according to any one of the preceding embodiments, wherein the cell population is derived from PSCs, preferably hPSCs.
30. The method according to embodiment 29, wherein obtaining the cell population comprises the step of allowing PSCs to differentiate into neural cells comprising neuron precursors.
31. The method according to embodiment 30, wherein the PSCs are differentiated into neural cells of the forebrain, midbrain, hindbrain, or spinal cord region.
32. The method according to any one of embodiments 30 and 31, wherein the PSCs are allowed to differentiate into neural cells for 15 days to 50 days.
33. The method according to any one of embodiments 30 and 31, wherein the PSCs are allowed to differentiate into neural cells for at least 20 days.
34. The method according to any one of embodiments 30 to 33, wherein the PSCs are contacted with at least one inhibitor of the SMAD signaling pathway.
35. The method according to any one of embodiments 30 to 34, wherein the cells are contacted with at least one activator of the WNT signaling pathway.
36. The method according to any one of embodiments 30 to 35, wherein the cells are contacted with an activator of SHH signaling pathway.
37. The method according to any one of embodiments 30 to 36, wherein the cells are contacted with FGF8.
38. The method according to any one of embodiments 30 to 37, wherein the cells are cultured on a substrate coated with a laminin or fragments thereof, preferably laminin-521 and/or laminin-511 and/or laminin-111.
39. The method according to any one of embodiments 30 to 38, wherein the cells are contacted with ascorbic acid.
40. The method according to any one of embodiments 30 to 39, wherein the cells are contacted with BDNF and/or GDNF.
41. The method according to any one of embodiments 30 to 40, wherein the cells are contacted with DAPT.
42. The method according to any one of the preceding embodiments, comprising the further step of maturing neurons and/or precursors thereof following the steps of selection and/or exclusion.
43. The method according to any one of the preceding embodiments, comprising the step of recovering the cell population enriched in neurons and/or precursors thereof following the steps of selection and/or exclusion and optionally following the further step of maturing the neurons and/or precursors thereof.
44. The method according to embodiment 43, wherein the recovered cell population enriched in neurons and/or precursors thereof is cryopreserved.
45. The method according to any one of embodiments 43 and 44, wherein the cells recovered are negative for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.
46. The method according to any one of the preceding embodiments, wherein the cell population enriched in neurons and/or precursors thereof has an increased propensity for developing into neurons.
47. The method according to any one of the preceding embodiments, wherein at least 50% of the neurons and/or precursors thereof are of the forebrain, midbrain, hindbrain, or spinal cord region.
48. The method according to any one of the preceding embodiments, wherein at least 50% of the neurons and/or precursors thereof are of the ventral midbrain region.
49. The method according to any one of embodiments 31 to 47, wherein at least 50% of the neurons and/or precursors thereof are of the forebrain region, and wherein the step of selecting and/or excluding is carried out between day 30 to 70, preferably between day 30 to 40.
50. The method according to any one of embodiments 31 to 47, wherein at least 50% of the neurons and/or precursors thereof are of the spinal cord region, and wherein the step of selecting and/or excluding is carried out between day 20 to 40, preferably between day 20 to 30.
51. The method according to any one of the preceding embodiments, wherein the selection and/or exclusion of the cells is carried out by antibody-mediated cell sorting.
52. The method according to any one of the preceding embodiments, wherein the selection and/or exclusion of the cells is carried out using fluorescent-activated cell sorting (FACS), magnetic-activated cell sorting (MACS), or immunopanning.
53. The method according to any one of the preceding embodiments, wherein the markers are surface markers.
54. The method according to any one of the preceding embodiments, wherein the cell population is in vitro.
55. The method according to any one of the preceding embodiments, wherein the cells of the cell population are non-native.
56. The method according to any one of the preceding embodiments, wherein the cell population is a neural cell population.
57. A cell population obtained by the method according to any one of the preceding embodiments.
58. A cell population of neurons and/or precursors thereof, wherein at least 50% of the cell population are positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or less than 50% of the cells are positive for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.
59. The cell population according to embodiment 58, wherein at least 50% of the cell population are positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and negative for one or more of the markers selected from SLIT2, NTN1, CMTM6, CMTM8, CMTM7, CRLF1, CD36, CD47, CD83, CD99, TNFRSF1A/CD120a, TNFRSF12a/CD266, TMEM123, LRP10, ITGB8, HLA-A, and RAMP1.
60. The cell population according to any one of embodiments 58 and 59, wherein at least 50% of the cell population are selected from ventral midbrain neurons and/or precursors thereof, ventral spinal cord neurons and/or precursors thereof, dorsal forebrain neurons and/or precursors thereof, and ventral forebrain neurons and/or precursors thereof.
61. The cell population according to any one of embodiments 58 to 60, wherein more than 80% of the cell population are negative for the marker CD47.
62. The cell population according to any one of embodiments 58 to 61, wherein more than 80% of the cell population are negative for the marker CD99.
63. The cell population according to any one of embodiments 58 to 62, wherein more than 80% of the cell population are negative for the markers CD47 and CD99.
64. The cell population according to any one of embodiments 58 to 63, wherein at least 60% of the cell population are positive for the marker FOXA2.
65. The cell population according to any one of embodiments 58 to 64, wherein at least 50% of the cell population are ventral midbrain neurons and/or precursors thereof.
66. The cell population according to any one of embodiments 58 to 65, wherein at least 80% of the cell population are positive for one or both of the markers ASCL1 and INA.
67. The cell population according to any one of embodiments 58 to 66, wherein the cell population has an increased propensity for developing into neurons.
68. The cell population according to any one of embodiments 58 to 67, wherein the cells of the cell population are in vitro differentiated cells.
69. The cell population according to any one of embodiments 58 to 68, wherein the cell population is in vitro.
70. The cell population according to any one of embodiments 58 to 69, wherein the cells of the cell population are non-native cells.
71. The cell population according to any one of embodiments 58 to 70, wherein the cells of the cell population are derived from hPSCs.
72. The cell population according to any one of embodiments 58 to 71, wherein the cells of the cell population are genetically modified.
73. The cell population according to embodiment 72, wherein the genetically modified cells are HLA-deficient.
74. The cell population according to any one of embodiments 58 to 73, wherein the cell population comprises at least 1,000 cells.
75. The cell population according to any one of embodiments 58 to 74, wherein the cell population is enriched in neurons and/or precursors thereof.
76. The cell population according to any one of embodiments 58 to 75, wherein the cell population is a neural cell population.
77. The cell population according to any one of embodiments 58 to 76 obtained by the method according to any one of embodiments 1 to 56.
78. A composition comprising a cell population according to any one of embodiments 57 to 77.
79. A cell population according to any one of the embodiments 57 to 77 or a composition according to embodiment 78 for use as a medicament.
80. The cell population or composition according to embodiment 79, for prevention or treatment of a condition requiring the administration of neurons and/or precursors thereof.
81. A cell population according to any one of embodiments 57 to 77 or a composition according to embodiment 78 for use in the treatment of a neurological condition selected from Parkinson's disease, stroke, traumatic brain injury, spinal cord injury, Huntington's disease, dementia, epilepsy, blindness, Alzheimer's disease, and other neurological conditions wherein neurons are lost or dysfunctional.
82. A method of treatment or prevention of a neurological condition comprising the administration to a patient of an effective amount of a cell population or a composition according to embodiment 79.

### EXAMPLES

The following are non-limiting examples for carrying out the invention.

### Example 1: Differentiation of Human Pluripotent Stem Cells to Neurons

Human embryonic stem cell (hESCs) lines RC17 (Roslin CT) and 3053 (Novo Nordisk A/S) were cultured in iPS Brew XF media (Miltenyi Biotec) supplemented with 60 U/mL Penicillin-Streptomycin (P-S; Thermo Fisher Scientific) on human laminin-521 matrix (0.7-1.2 μg/cm²; Biolamina) coated culture ware. Media was changed daily, and cells passaged with EDTA 0.5 mM (Thermo Fisher Scientific) every 4-6 days. Cultures were maintained at 37° C., humidity 95% and a 5% CO₂ level.

hESCs were differentiated to ventral midbrain neurons according to an established protocol (Nolbrant et al., 2017; Kirkeby et al., 2017). In brief, hESC were grown to 70-90% confluency, then disassociated with 0.5 mM EDTA. The cells were seeded at 10⁴ cells/cm² in cell culture flasks or plates coated with human laminin-111 (1.2 μg/cm²; BioLamina) and immediately put into contact with differentiation media. The cells were exposed to N2-based media from days in vitro (DIV) 0-8; 50% DMEM/F12+Glutamax (Gibco), 50% Neurobasal (Gibco), 1% N2 supplement CTS (Thermo Fisher Scientific), 5% GlutaMAX (Thermo Fisher Scientific), 0.2% P-S (Thermo Fisher Scientific) and supplemented with SMAD inhibitors SB431542 (10 μM; Miltenyi Biotec), Noggin (100 ng/mL; Miltenyi Biotec) for neural induction, Sonic Hedgehog C24II (SHH; 500 ng/mL; Miltenyi Biotec) for ventral fate, GSK3β inhibitor CHIR99021 (CHIR; 0.5-0.6 μM; Miltenyi Biotec) to promote caudalisation. N2-based media was supplemented with fibroblast growth factor 8b (FGF8b; 100 ng/mL; Miltenyi Biotec) from DIV9-11. At DIV11, the cells were dissociated with accutase (Thermo Fisher Scientific) and seeded at 0.8×10⁶ cells/m² in a cell culture flask or plate coated with human laminin-111 (1.2 μg/cm²) in DIV11-16 media (Neurobasal, 2% B27 supplement without vitamin A CTS (Thermo Fisher Scientific), 5% GlutaMAX, 0.2% P-S and supplemented with FGF8b (100 ng/mL), L-ascorbic acid (AA; 200 μM; Sigma), human Brain Derived Neurotrophic Factor (BDNF; 20 ng/mL; Miltenyi Biotec)) supplemented with Y-27632 (Miltenyi Biotec) at 10 μM. At day in vitro (DIV) 16, the cells were dissociated with accutase and either cryopreserved, or re-seeded in cell culture flasks/plates coated with poly-L-ornithine (0.002%) and Laminin-521 (1.5 μg/cm²) in B27 media supplemented with BDNF (20 ng/mL), GDNF (20 ng/mL), L-ascorbic acid (200 μM), dcAMP (500 μM), DAPT (10 μM), and Y-27632 (10 μM) for extended in vitro culture allowing further differentiation and maturation of ventral midbrain neural stem cells into neurons.

### REFERENCES

Nolbrant S, Heuer A, Parmar M, Kirkeby A. Generation of high-purity human ventral midbrain dopaminergic progenitors for in vitro maturation and intracerebral transplantation. Nat Protoc. 2017 September; 12(9):1962-1979. doi: 10.1038/nprot.2017.078. Epub 2017 Aug. 31. PMID: 28858290.
Kirkeby A, Nolbrant S, Tiklova K, Heuer A, Kee N, Cardoso T, Ottosson D R, Lelos M J, Rifes P, Dunnett S B, Grealish S, Perlmann T, Parmar M. Predictive Markers Guide Differentiation to Improve Graft Outcome in Clinical Translation of hESC-Based Therapy for Parkinson's Disease. Cell Stem Cell. 2017 Jan. 5; 20(1):135-148. doi: 10.1016/j.stem.2016.09.004. Epub 2016 Oct. 27. PMID: 28094017; PMCID: PMC5222722.

### Example 2: scRNA Sequencing Experimental Design to Identify Novel Selection Candidates and Technical Methodology

To perform single cell RNA sequencing (scRNA-seq), cell cultures were dissociated into single cell suspensions with accutase, and 3000-10000 cells were processed using the 10X Genomics Chromium Platform and sequenced on a NextSeq550. Data was curated to filter out apoptotic and stressed as well as multiplets.

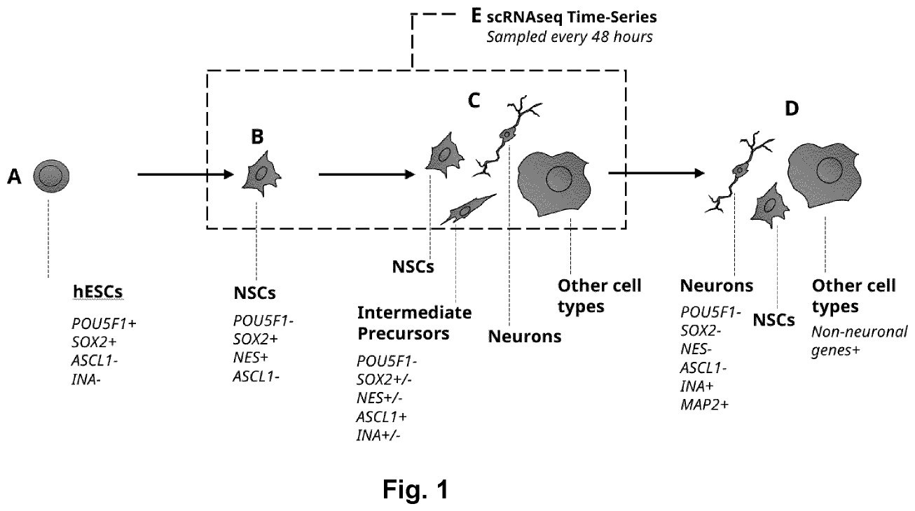

Differentiating human pluripotent stem cells (hPSCs) to ventral midbrain dopamine neurons following established protocols causes cells to transition through several cardinal cell stages of development. Firstly, hPSCs transition to neural stem cells (NSCs) (FIG. 1 A-B), then subsequently intermediate precursor cells (IPs) (FIG. 1 C) and finally to terminally differentiated cell types (FIG. 1 D). This process is not precise, however, and following the NSC stage cultures lose their homogeneity and typically begin to differentiate into a diversity of cell types and form a mixed cell culture (FIG. 1 C). This mixed culture composition persists until differentiation is completed and cells acquire a terminal fate (FIG. 1 D). Different expression combinations of genes (typically cell fate instructing transcription factor proteins) are used to identify these varied cell types and are shown in FIG. 1 A-D.

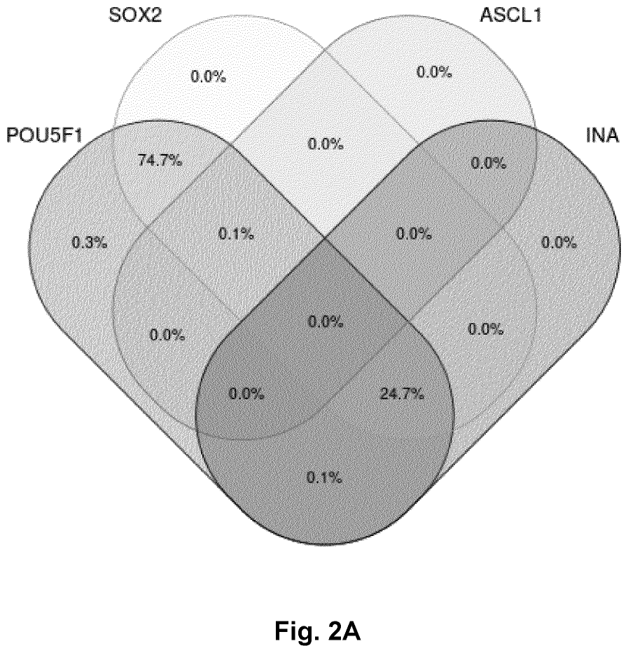

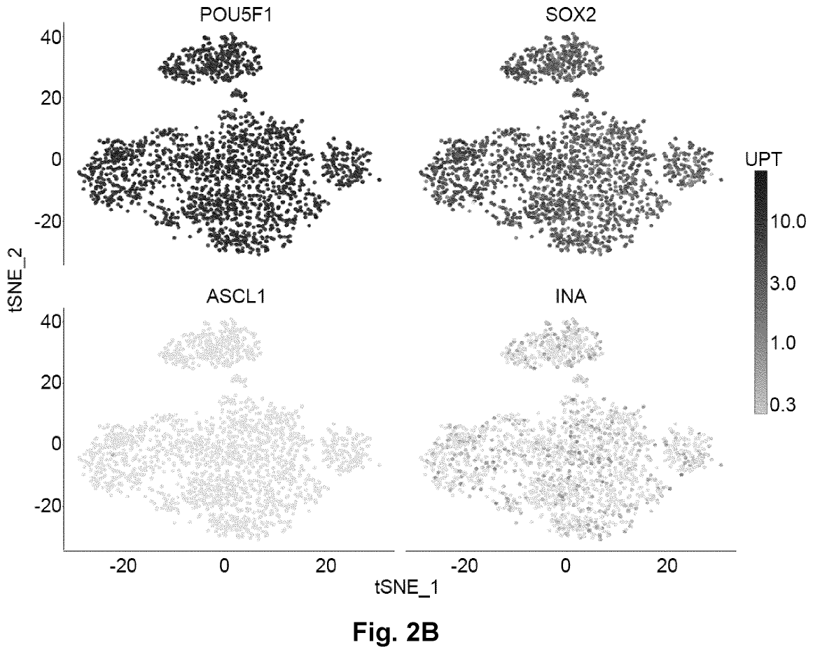

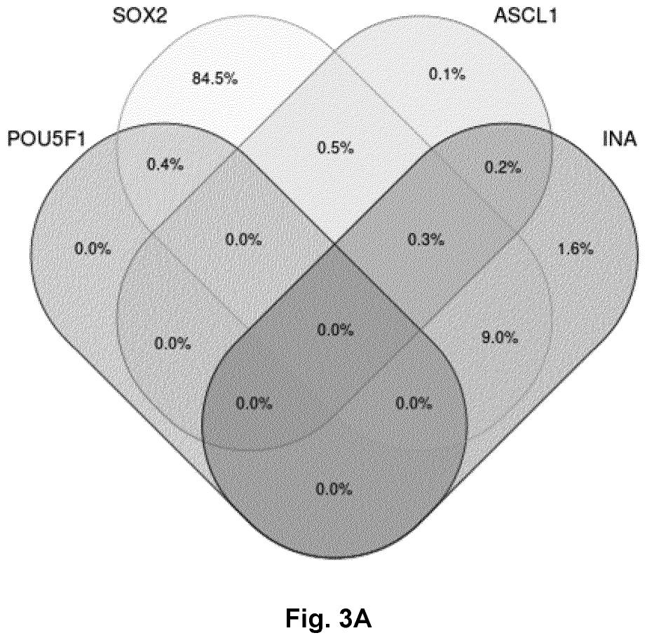

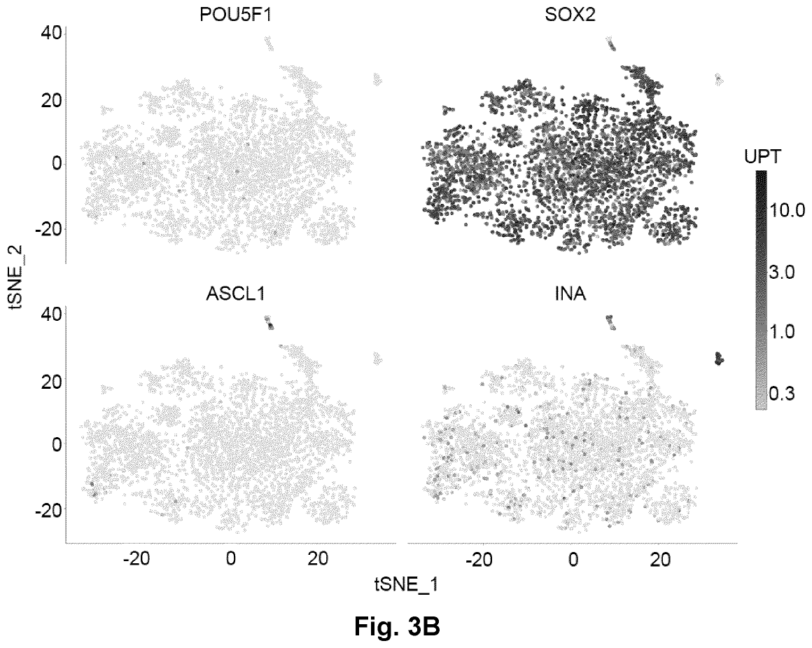

Single cell RNA-sequencing (scRNA-seq) analysis was applied at several timepoints along the differentiation allowing the observation and monitoring of the transition from homogeneous cultures to heterogeneous mixed cultures. hPSCs used for differentiation and the NSCs they form are highly homogeneous as seen by their transcriptomic profiles. hPSCs express markers of their pluripotent identity in high levels, with 99.5% of hPSCs analysed expressing the pluripotency marker POU5F1 and pluripotency/NSC marker SOX2 and these genes are expressed at high levels (FIG. 2A, 2B). Furthermore, hPSCs express minimal or low levels of other genes indicative of a differentiated cell fate, such as the intermediate precursor (IP) marker ASCL1 or neuronal marker INA (FIG. 2A, 2B). Similarly, NSCs are highly homogenous, expressing the NSC marker SOX2, but do not express the pluripotency marker POU5F1, and have minimal and low expression of the IP marker ASCL1 and neuronal marker INA (FIG. 3A, 3B).

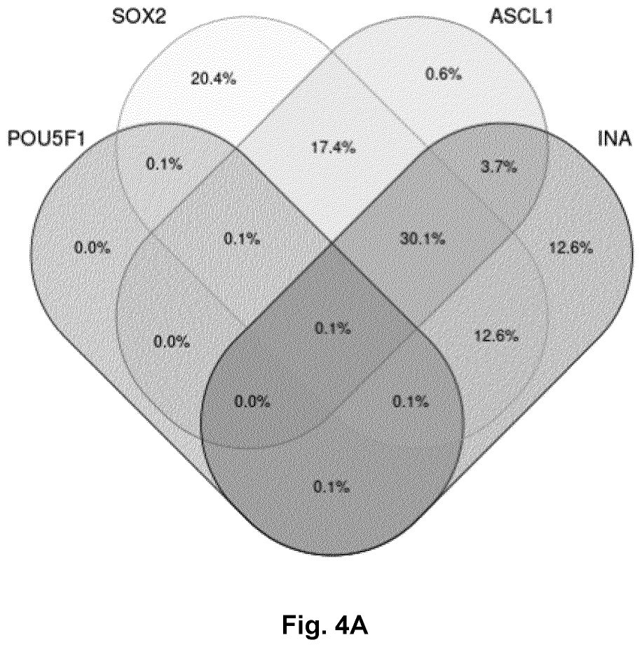

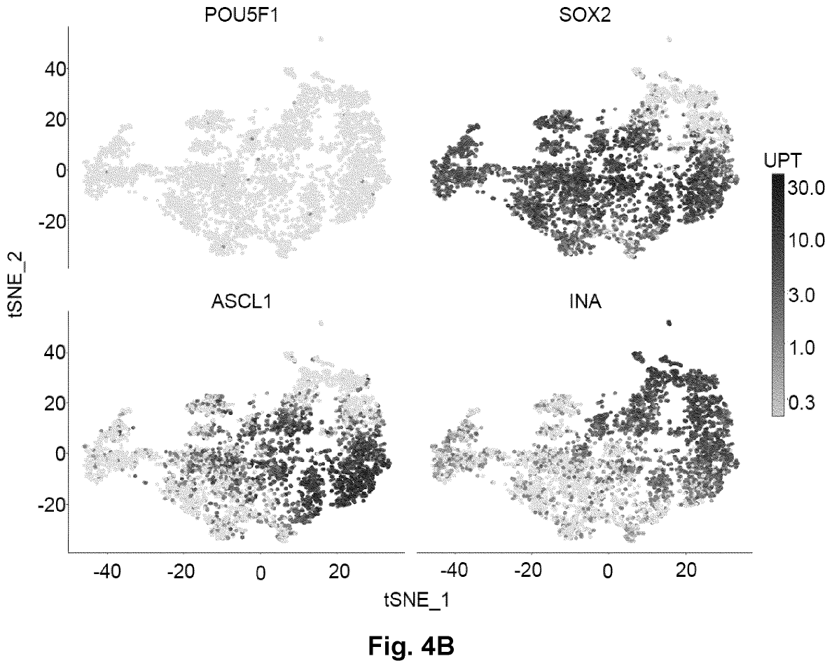

The heterogeneity that emerges in cultures after the NSC stage is evident at the transcriptional level and is shown as an example of scRNA-seq analysis at DIV24 (FIG. 4A, 4B). At this timepoint, the cell population expresses markers of various cell types and stages, for example NSCs at 20.4% (POU5F1−/SOX2+/ASCL1−/INA− cells; FIG. 4A) as well as intermediate precursor cells at 48.2% (POU5F1−/SOX2+ or −/ASCL1+/INA+ or −; FIG. 4A) and neurons at 16.3% (POU5F1−/SOX2−/ASCL1+ or −/INA+ or −; FIG. 4A). Pluripotent stem cells are not present at this stage due to their differentiation into NSCs, IPs and Neurons (POU5F1+/SOX2+/ASCL1−/INA−; FIG. 4A, 4B).

### Example 3: scRNA Sequencing of Mixed Culture Stage and Identification of Cell Populations and Purification Candidates

A scRNA-seq time-course study was performed with sampling every 48 hours from the point at which cells are homogenous (DIV16 NSCs) to when they have developed into mixed cultures (DIV26 mixed cultures; FIG. 1 E). Sampling every 48 hours within this window (DIV16, 18, 22, 24, 26) ensured a continuum of data was obtained to enable the trajectories of cell differentiation and diversification into different cell stages and types to be observed and mined for markers that may be of use to separate populations of interest or disinterest with surface antigens restricted to these populations.

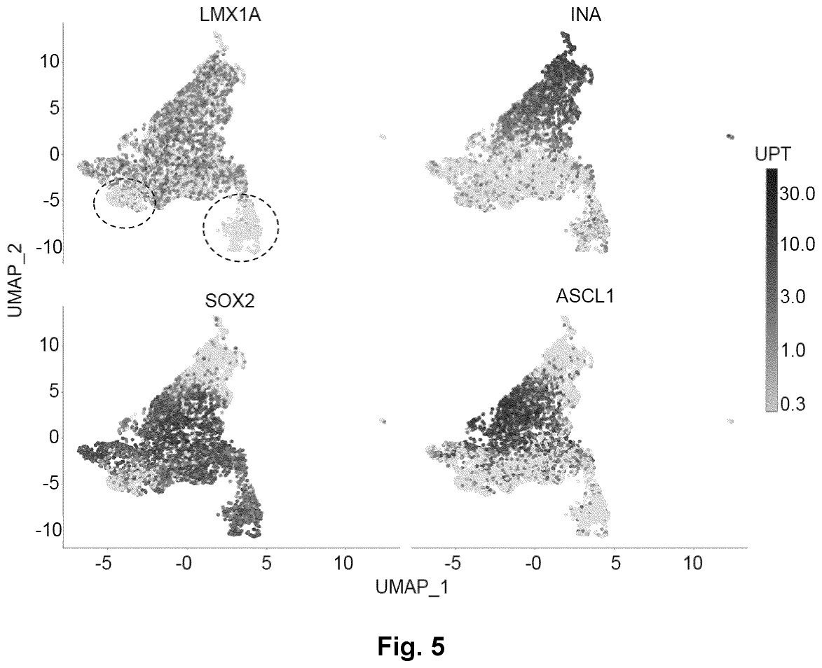

We identified mixed cultures between DIV22-26 and aligned these datasets to search for subtype-restricted surface antigens (FIG. 5). Here, every cell (represented by a single dot) can be seen to express genes corresponding to different cell stages such as NSCs and off-targets which were present predominantly in the bottom half of the t-SNE plot (SOX2+; FIG. 5), IPs located at the middle of the t-SNE plot (ASCL1+; FIG. 5) and neurons located at the top of the t-SNE plot (INA+; FIG. 5). Further indicating the heterogeneity of these cultures, a marker corresponding to the ventral midbrain floor plate region of the embryo (LMX1A) was not expressed in all cells and selectively absent in two clusters at the bottom of the t-SNE plots (FIG. 5, circled). By comparing clusters that corresponded to these different cell types and stages, we were able to identify genes restricted in their expression to these identities.

### Example 4: Expression Profiles of Candidate Genes for NSC, IP and Neuron Isolation

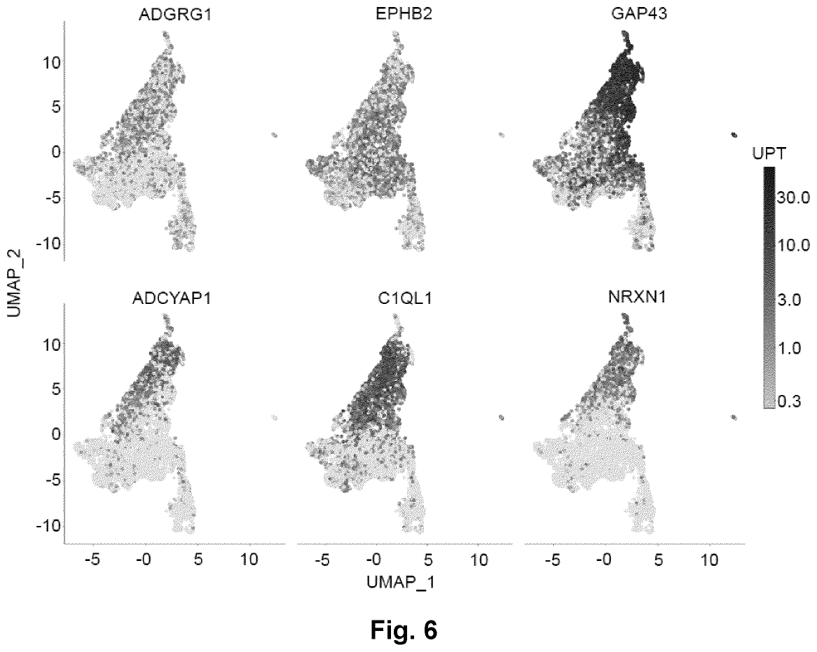

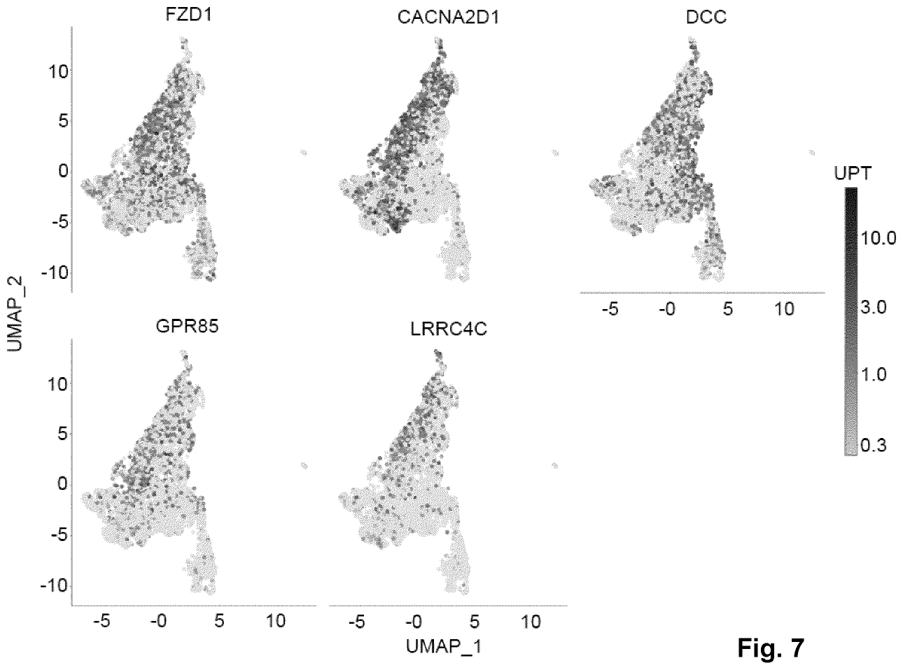

The highest scored differentially expressed surface marker genes in neuronal clusters were confirmed to have distributions throughout the t-SNE plot similar to the neuronal marker INA (FIG. 6-7), and these genes are deemed suitable for the isolation of neurons from mixed cultures. This includes the genes ADGRG1, EPFHB2, GAP43, ADCYAP1, C1QL1 and NRXN1 (FIG. 6) as well as FZD1, CACNA2D1, DCC, GPR85 and LRRC4C.

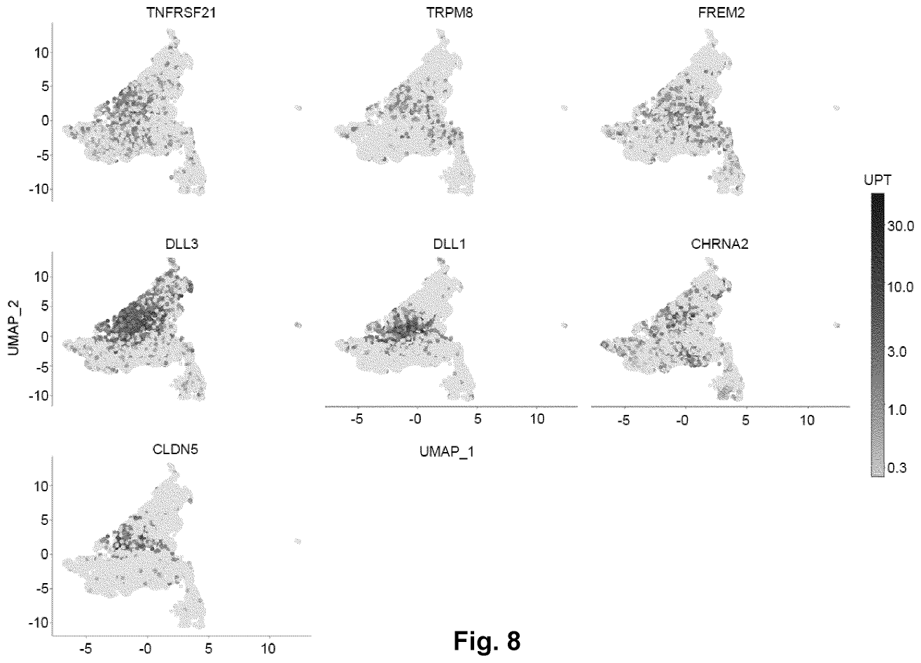

The highest scored differentially expressed surface marker genes in IP cell clusters were confirmed to have distributions throughout the t-SNE plot similar to the IP cell marker of the dopamine neuron lineage ASCL1 (FIG. 8), and these genes are deemed suitable for the isolation of neurons from mixed cultures. This includes the genes TNFRSF21, TRPM8, FREM2, DLL3, DLL1, CHRNA5 and CLDN5.

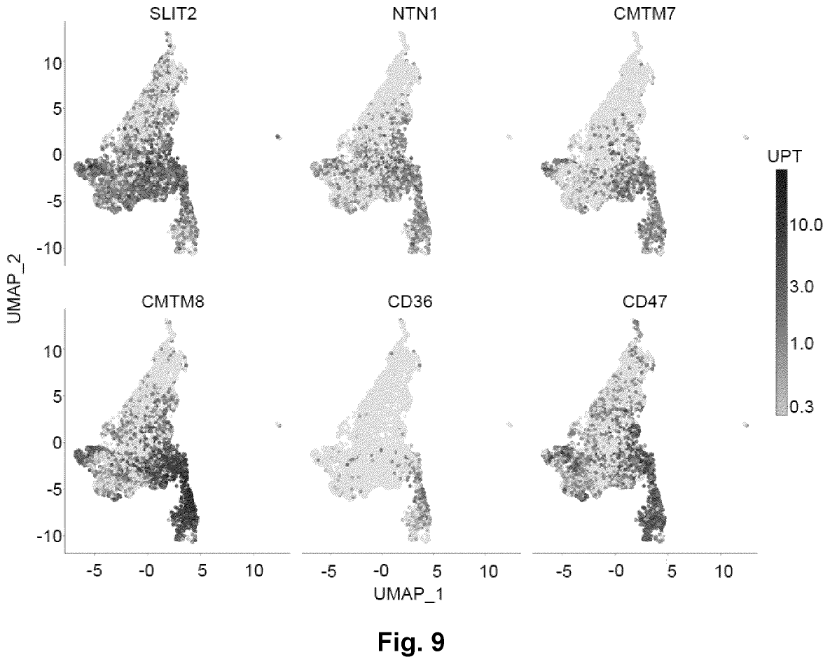

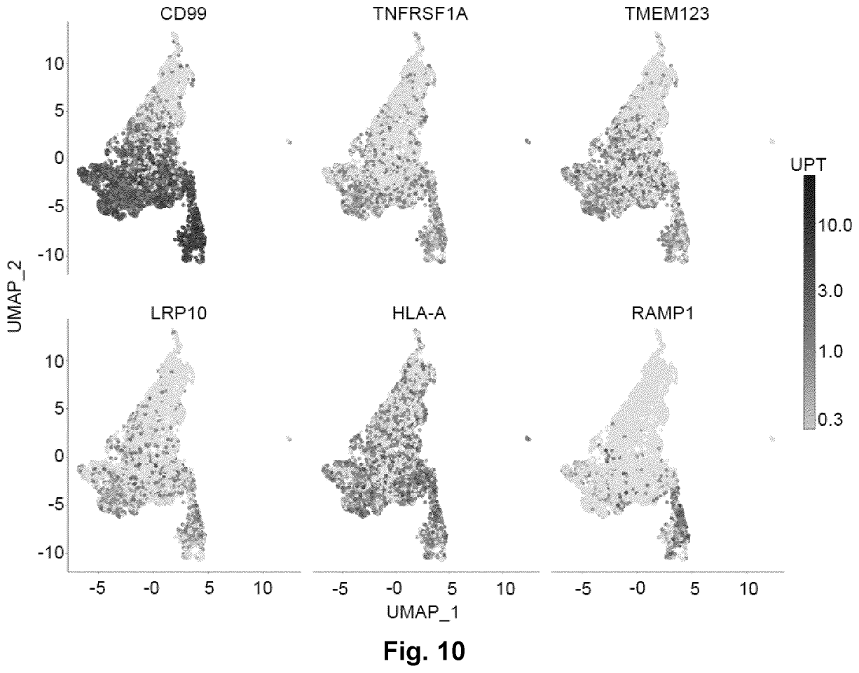

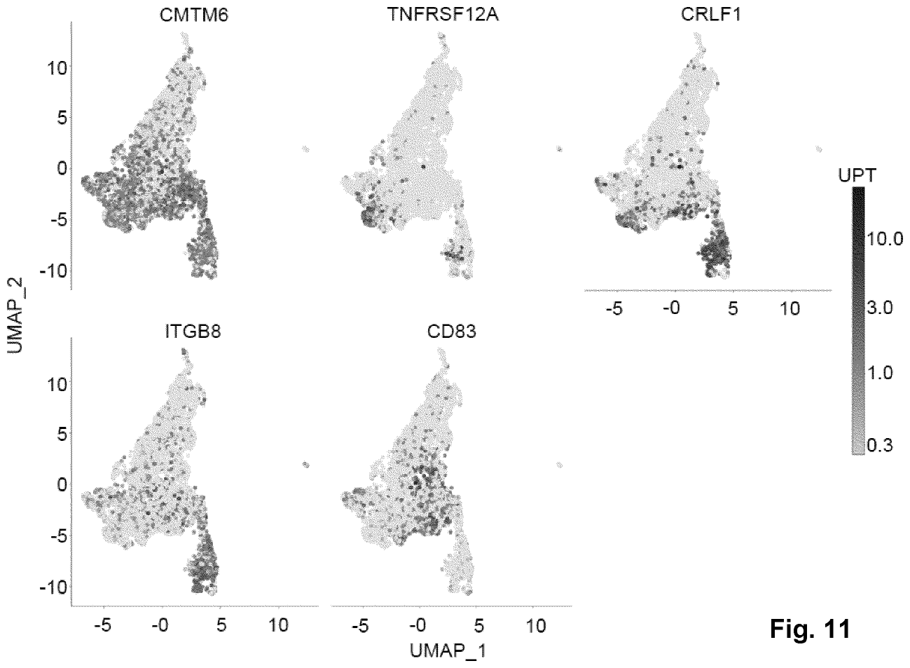

The highest scored differentially expressed surface marker genes in NSC/Off-target clusters were confirmed to have distributions throughout the t-SNE plot similar to the NSC/Off-target marker SOX2 (FIG. 9-11), and these genes are deemed suitable for the isolation of NSC/Off-target cells from mixed cultures. This includes the genes SLIT2, NTN1, CMTM7, CMTM8, CD36, CD47, CD99, TNFRSF1A, TMEM123, LRP10, HLA-A, RAMP1, CMTM6, TNFRSF12A, CRLF1, ITGB8 and CD83.

### Example 5: Fluorescence-Activated Cell Sorting (FACS) of Midbrain Neural Cells Using Antibodies Against CD47 and CD99

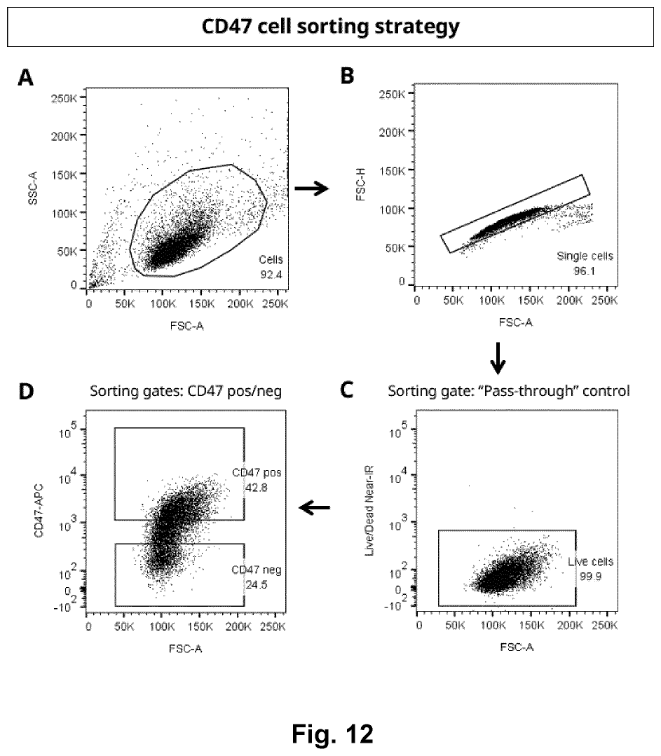

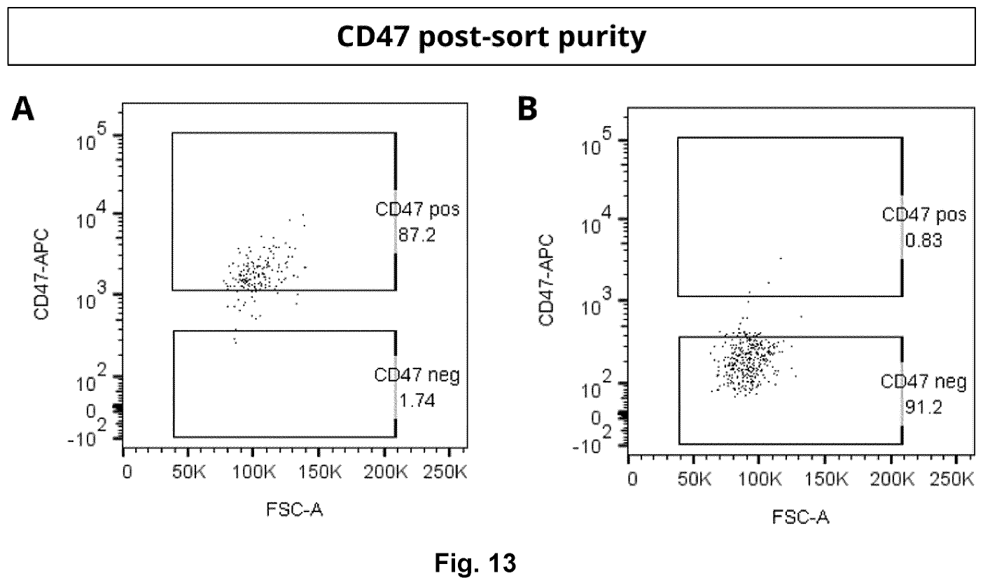

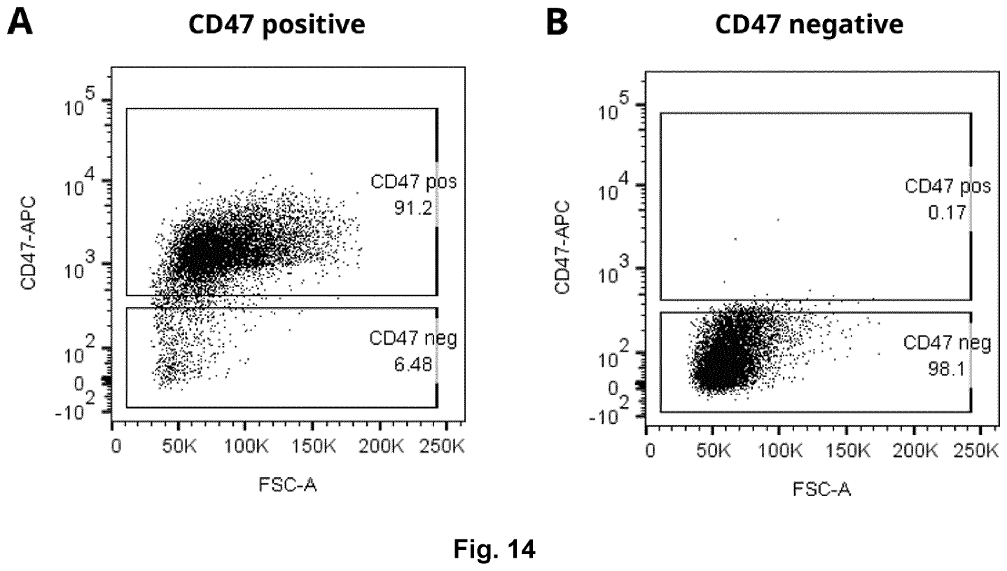

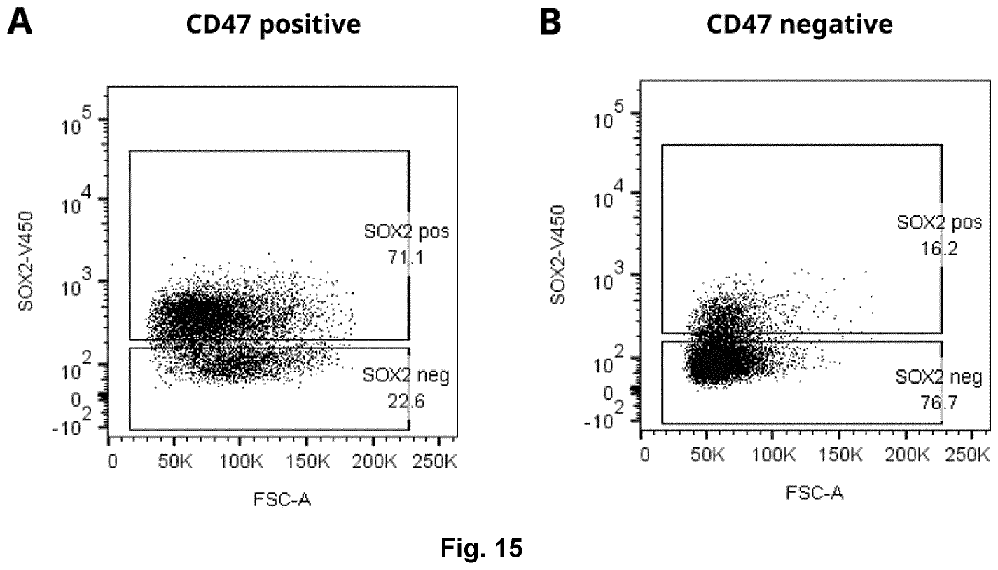

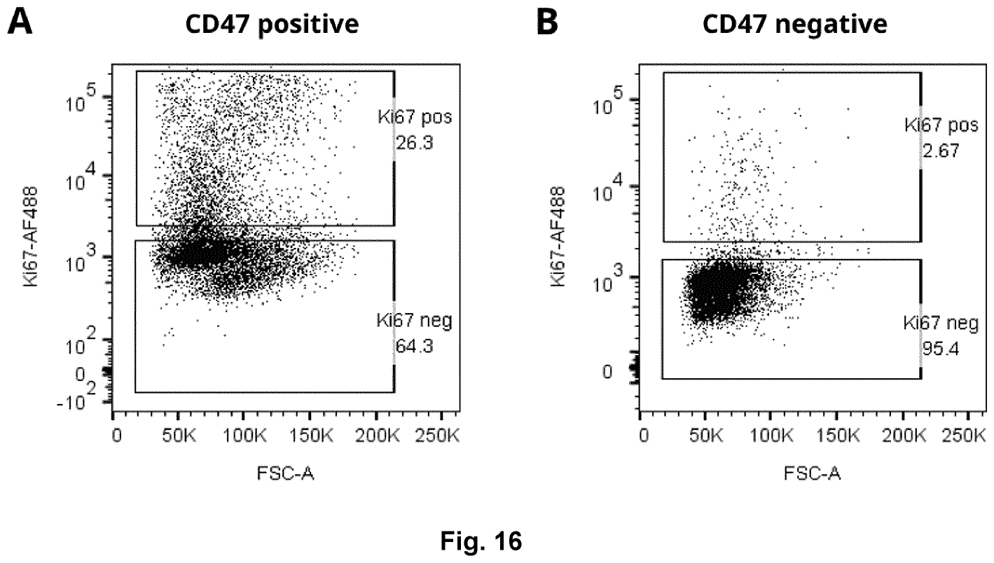

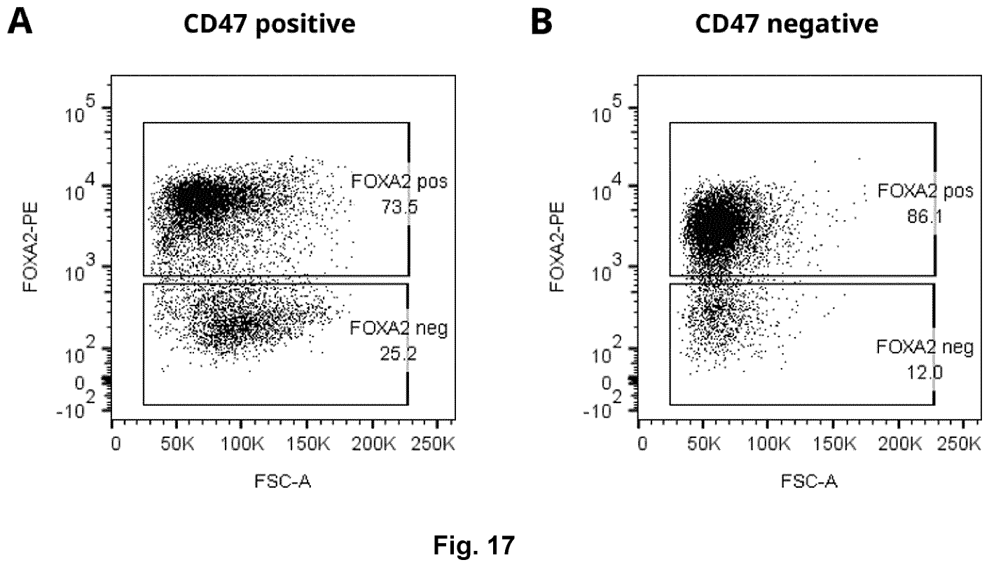

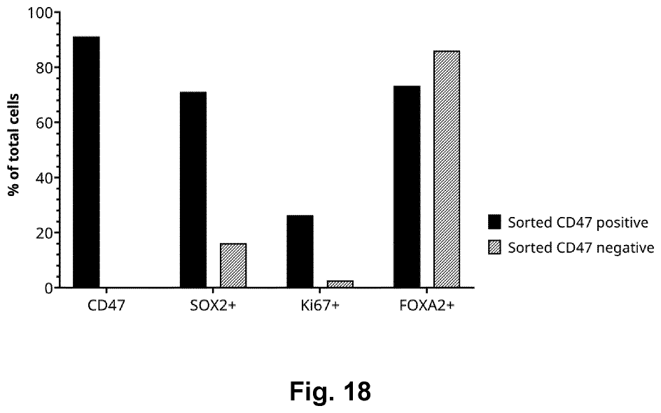

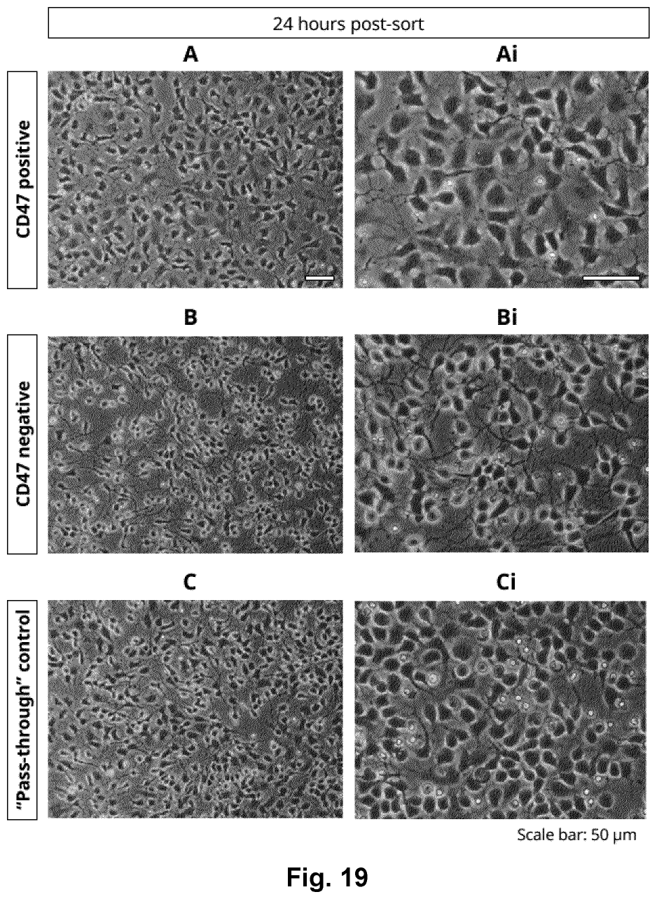

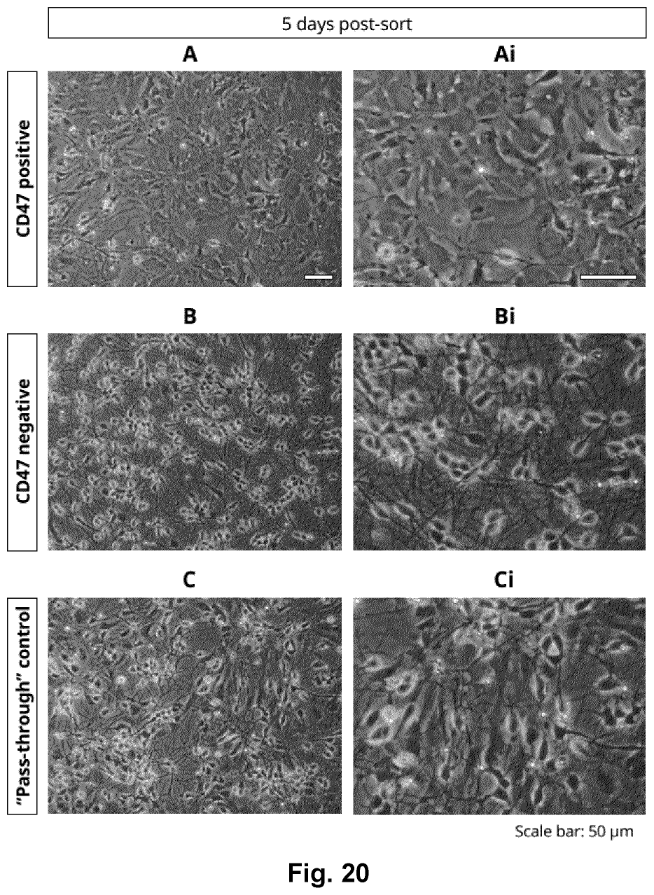

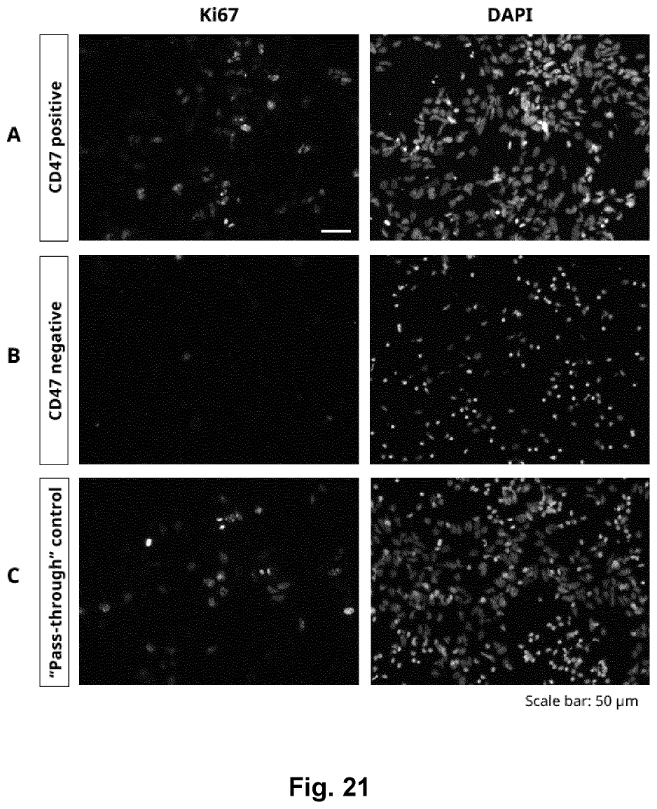

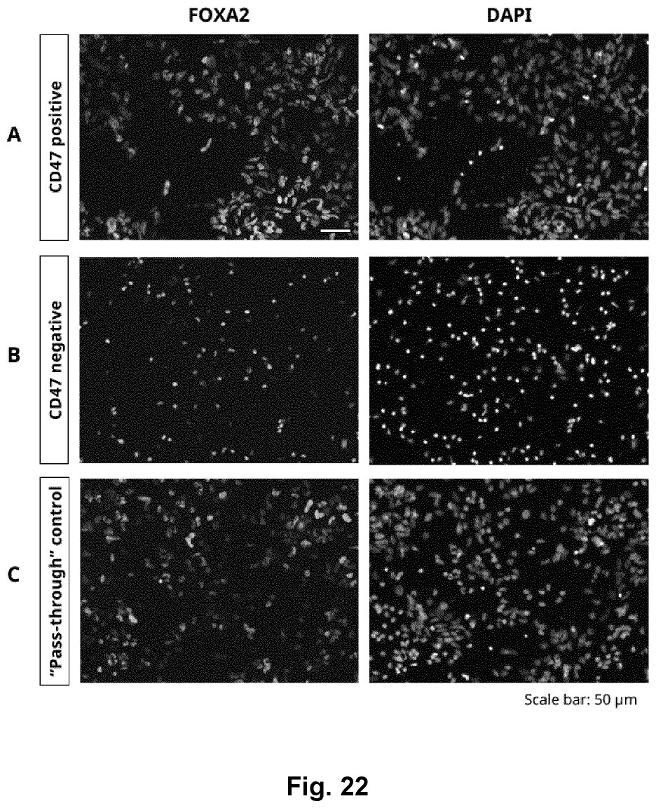

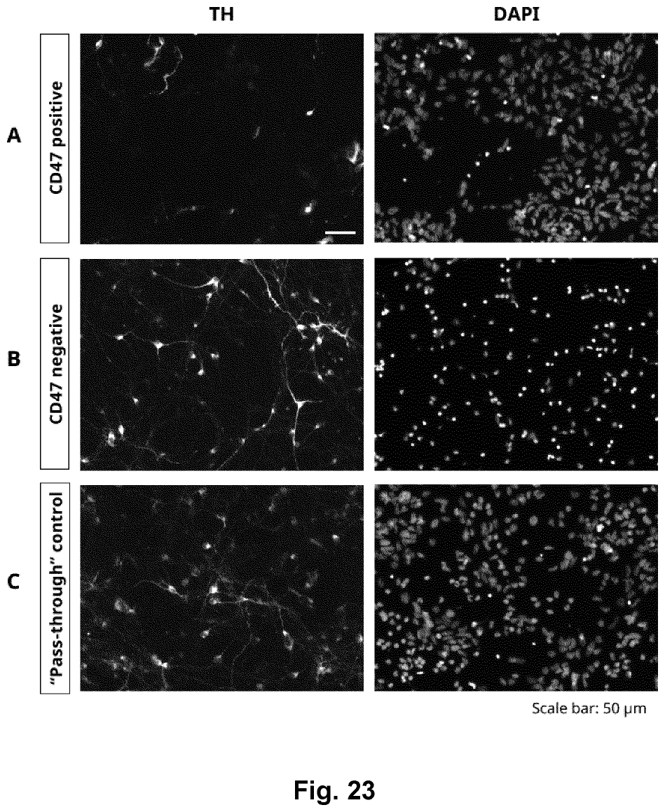

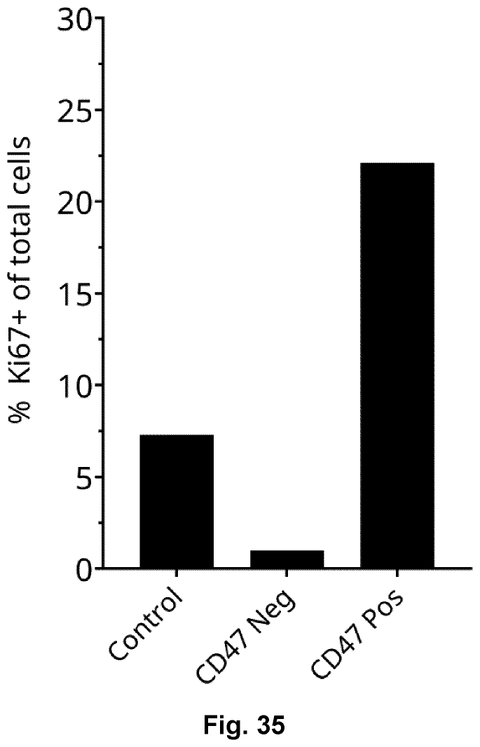

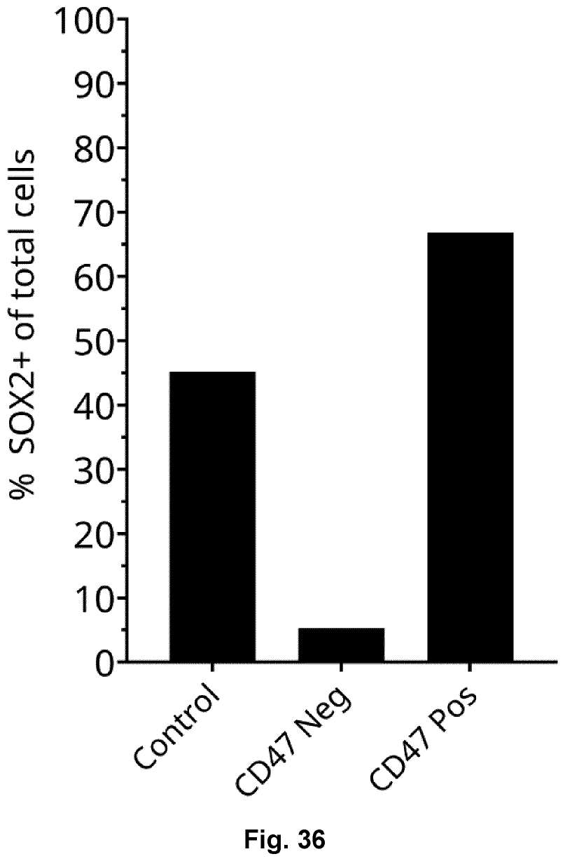

Ventral midbrain dopaminergic (vmDA) progenitor cells were generated from hESCs in 2D in vitro culture as described in example 1. At 25 days after initiating the differentiation, the cell culture was dissociated into a single cell suspension using accutase and collected in N2 media (CTS™ Neurobasal™ medium supplemented with 1% CTS™ N-2 supplement). Dead cells were labelled using a LIVE/DEAD™ Fixable Near-IR Dead Cell Stain Kit. The cells were then resuspended in B27 media (CTS™ Neurobasal™ medium supplemented with 1% B-27™ supplement without vitamin A, 2 mM GlutaMAX™, 60 U/mL Penicillin-Streptomycin, 10 μM ROCK inhibitor) and stained using an APC-conjugated antibody against CD47 at a concentration of 1:2500. CD47 positive and negative cells were purified by fluorescence-activated cell sorting (FACS) using a BD FACSAria™ Fusion instrument (FIG. 12). A “pass-through” control sample was obtained by sorting the total live cell population (FIG. 12 C). After the FACS, the purity was confirmed to be >87% and >91% for the CD47 positive and negative cell fractions, respectively (FIG. 13). Immediately after the FACS, the sorted cells were re-seeded in 96-well plates coated with poly-L-ornithine (0.002%) and Laminin-521 (1.5 μg/cm²) in B27 media supplemented with BDNF (20 ng/mL), GDNF (20 ng/mL), L-ascorbic acid (200 μM), dcAMP (500 μM), DAPT (10 μM), and Y-27632 (10 μM) at a density of 450,000 cells/cm². Phase-contrast images acquired after 24 hours (FIG. 19) and 5 days (FIG. 20) showed high viability of the sorted cells and clear differences in morphology between the CD47 positive and negative cells; the CD47 positive cells appear to have large, low-contrast cell bodies with few or no projections (FIGS. 19 A-Ai, 20 A-Ai), while the CD47 negative cells display typical neuronal morphology characterized by small, high-contrast, round/oval cell bodies with well-defined projections, which are seen to increase in number and length over the five-day culture period (FIGS. 19 B-Bi, 20 B-Bi). As expected, the “pass-through” control sample contains a mixture of cells with these two distinct morphologies (FIGS. 19 C-Ci, 20 C-Ci). Furthermore, immediately after cell sorting, samples of the sorted cells were processed for intracellular flow cytometry using the BD Transcription Factor Buffer Set, stained with antibodies against SOX2 (1:30), Ki-67 (1:100) and FOXA2 (1:320), recorded using a BD FACSymphony™ flow cytometer, and analysed using FlowJo 10.7.2 software. Firstly, the purity of the sorted samples was confirmed to be >91% (FIG. 14, 18). The CD47 positive sorted fraction was found to be enriched for cells expressing the neural stem cell marker SOX2 (71.1%; FIG. 15 A, 18) as well as the proliferation marker Ki-67 (26.3%; FIG. 16 A, 18), while the CD47 negative sorted fraction showed markedly lower levels of both SOX2 (16.2%; FIG. 15 B, 18) and Ki-67 (2.67%; FIG. 16 B, 18), corresponding to a difference in the percentage of cells expressing SOX2 and Ki67 of 77.2% and 89.8%, respectively. These results support the hypothesis that CD47 antibody-based selection can be used for enrichment of neurons and depletion of proliferative neural stem cells and their derivatives without depleting the lineage of interest as seen by the percentage of cells expressing FOXA2, a marker of the developing floor plate (FIGS. 17, 18). These results were supported by immunofluorescence (IF) staining of the cells re-seeded in vitro. At 5 days post-sort, the cells were fixed in 4% paraformaldehyde and stained with primary antibodies against Ki-67 (1:250), FOXA2 (1:100), and tyrosine hydroxylase (TH; 1:500) followed by fluorescently labelled secondary antibodies. Images were acquired on an Olympus IX81 microscope using CellSens software. The IF staining confirmed the flow cytometry results showing that enrichment of CD47 negative cells reduces the number of Ki67+ proliferative cells (FIG. 21, FIG. 35) and SOX2+ cells (FIG. 36) without depleting the cells of interest as seen by FOXA2 and the dopamine neuron-specific marker TH (FIGS. 22, 23).

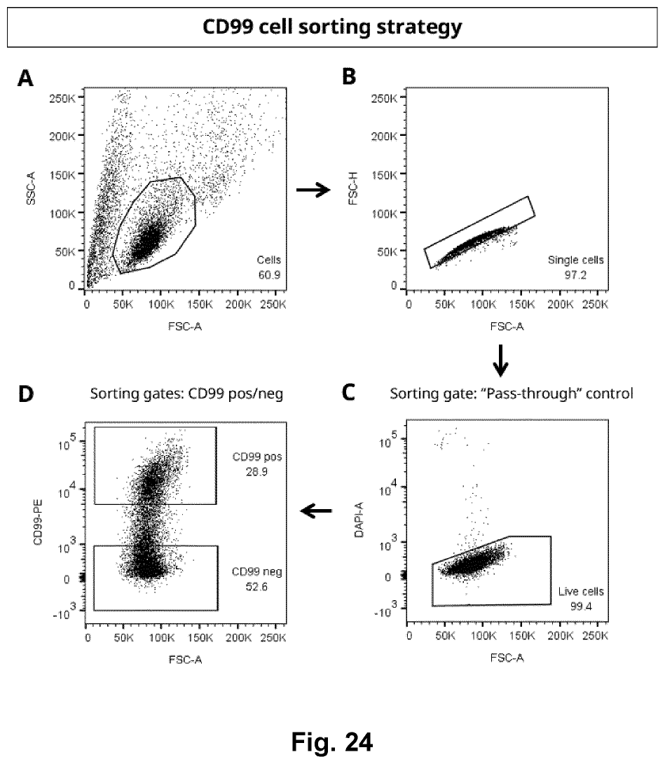

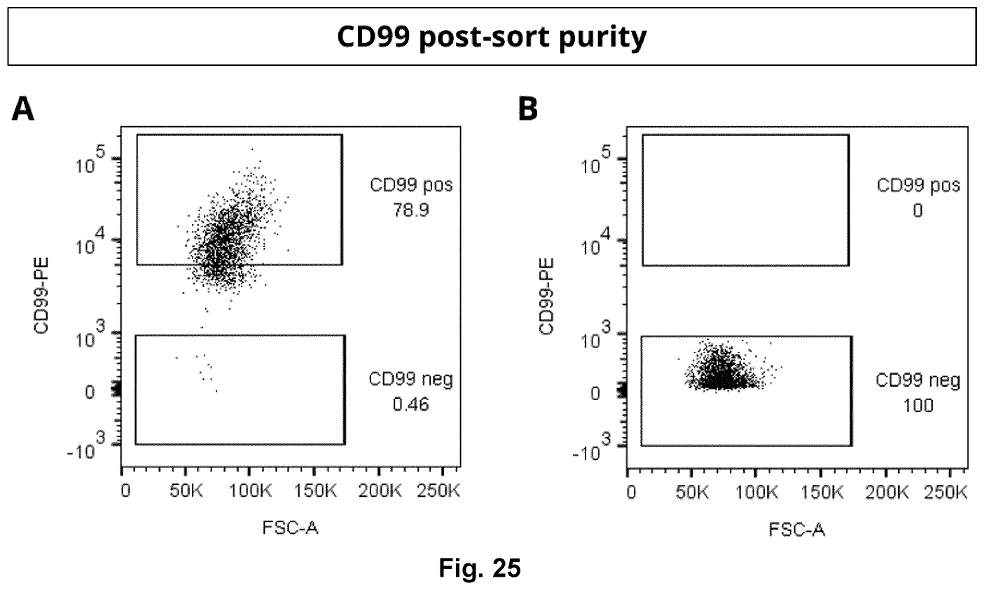

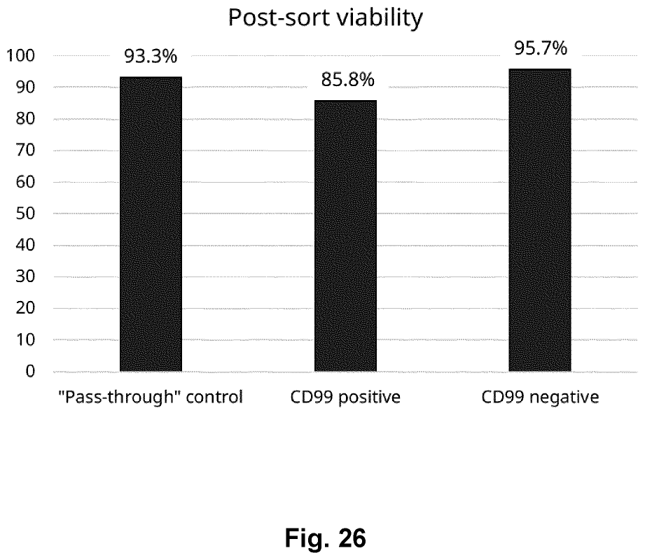

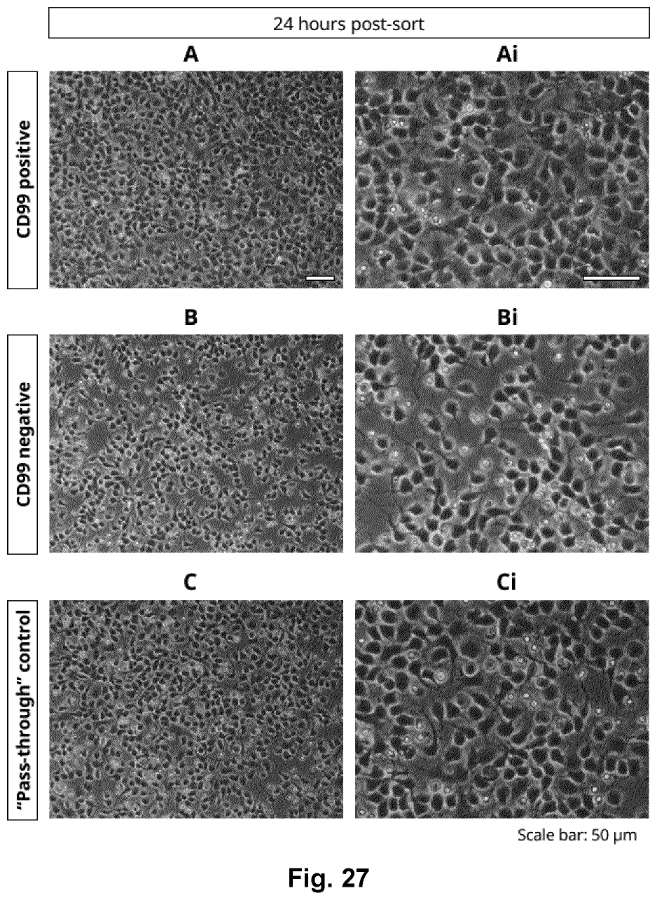

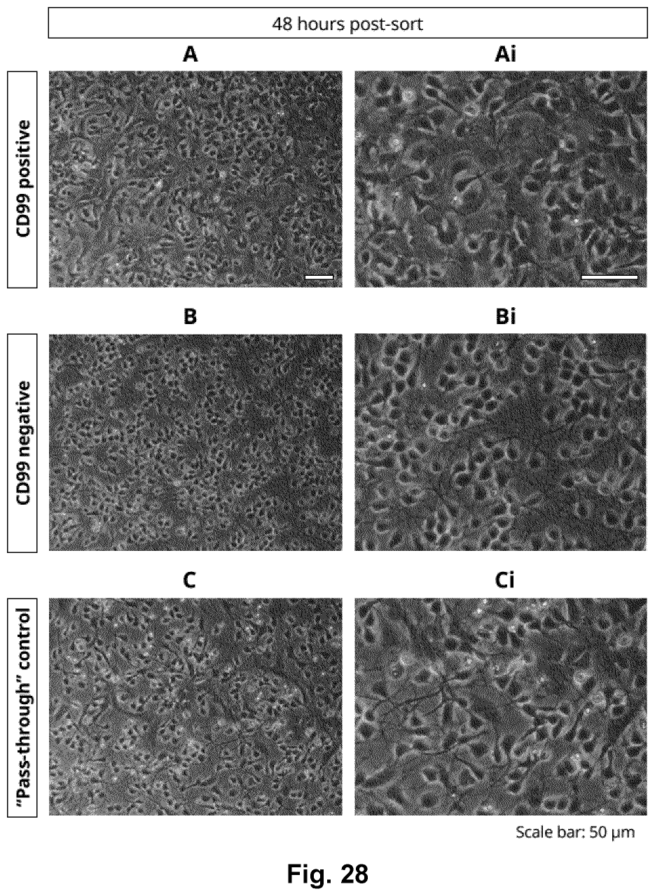

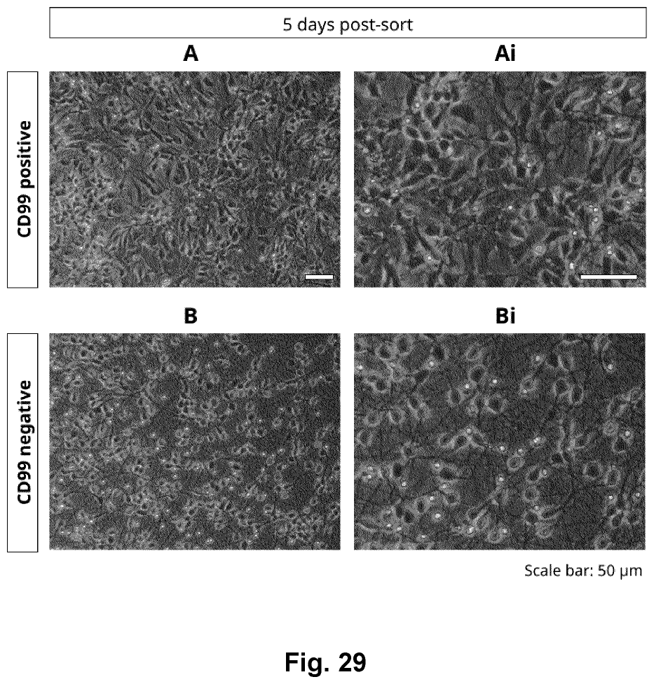

In a similar experiment, day 25 vmDA cultures were dissociated into a single cell suspension and stained using a PE-conjugated antibody against CD99 at a concentration of 1:500. CD99 positive and negative cells were purified by fluorescent-activated cell sorting (FACS) using a BD FACSAria™ Fusion instrument (FIG. 24). A “pass-through” control sample was obtained by sorting the total live cell population (FIG. 24 C). After the FACS, the purity was confirmed to be 79% and 100% for the CD99 positive and negative cell fractions, respectively (FIG. 25), and high viability (>85%) was observed in all sorted fractions (FIG. 26). The sorted cells were re-seeded in 96-well plates coated with poly-L-ornithine (0.002%) and Laminin-521 (1.5 μg/cm²) in B27 media supplemented with BDNF (20 ng/mL), GDNF (20 ng/mL), L-ascorbic acid (200 μM), dcAMP (500 μM), DAPT (10 μM), and Y-27632 (10 μM) at a density of 450,000 cells/cm². Phase-contrast images acquired after 24 hours (FIG. 27), 48 hours (FIG. 28), and 5 days (FIG. 29) showed good recovery of the sorted cells and clear differences in morphology between the CD99 positive and negative cells; the CD99 negative fraction was found to contain non-neuronal cells characterized by large, low-contrast cell bodies and few or no projections (FIGS. 27 A-Ai, 28 A-Ai, 29 A-Ai). These non-neuronal cells were absent in the CD99 negative sorted fraction, in which the cells instead displayed typical neuronal morphology characterized by small, high-contrast, round/oval cell bodies with well-defined projections, which are seen to increase in number and length over the five-day culture period (FIGS. 27 B-Bi, 28 B-Bi; 29 B-Bi). As expected, the “pass-through” control sample contains a mixture of cells with these two distinct morphologies (FIGS. 27 C-Ci, 28 C-Ci).

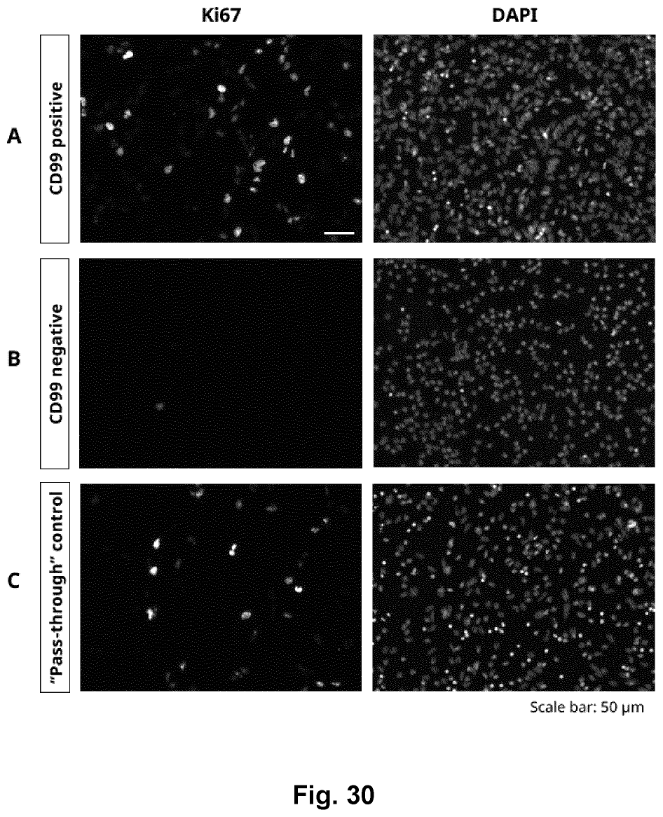

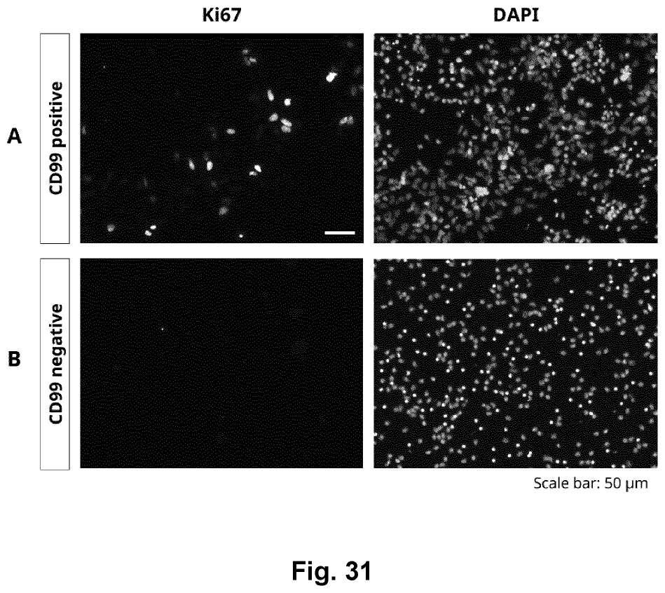

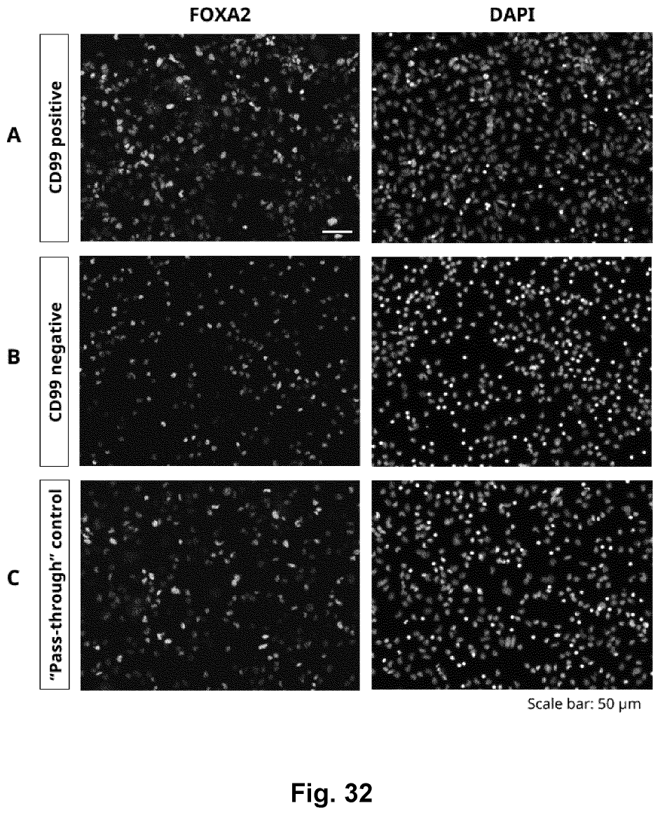

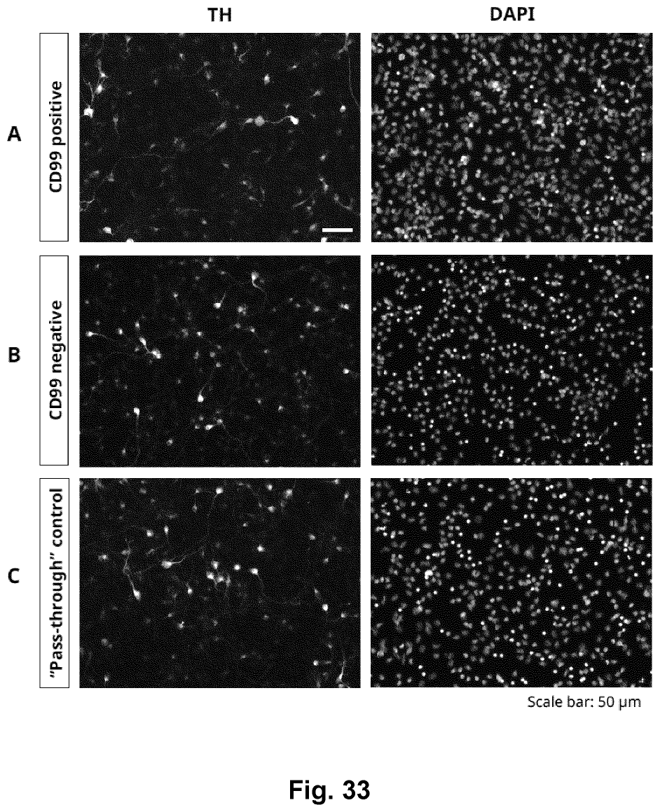

At 48 hours and at 12 days post-sort, the cells were assessed qualitatively by IF with antibodies against Ki-67, FOXA2, and TH. The IF staining showed that the CD99 negative sorted fraction contained a reduced number of cells expressing Ki-67 compared to the CD99 positive and “pass-through” control cells (FIGS. 30, 31), while still displaying large amounts of cells expressing the dopamine neuron lineage-specific markers FOXA2 and TH (FIGS. 32, 33). Together, these results support the hypothesis that CD99 antibody-based selection can be used for enrichment of neurons and depletion of proliferative neural stem cells and their non-neuronal derivatives.

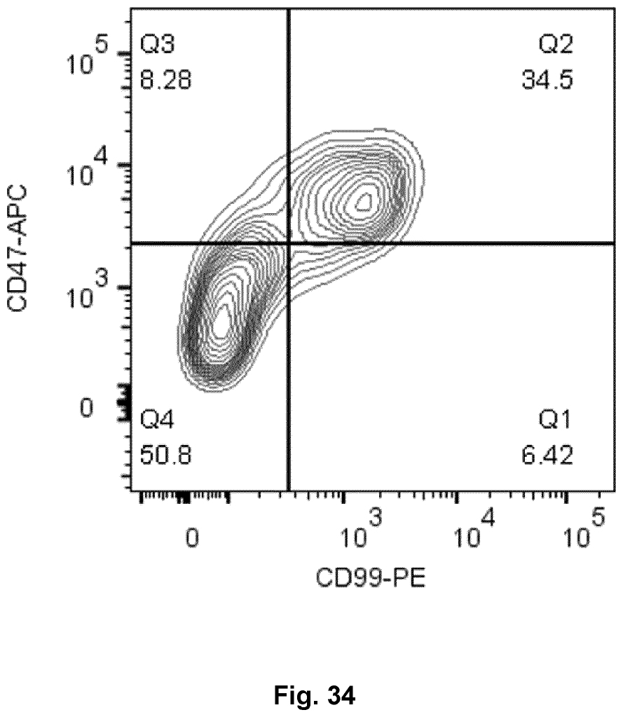

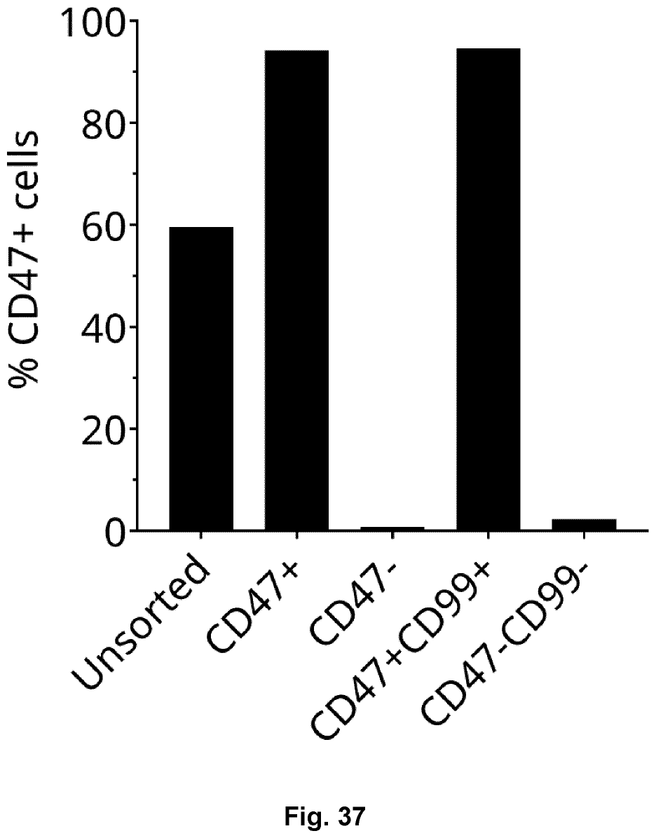

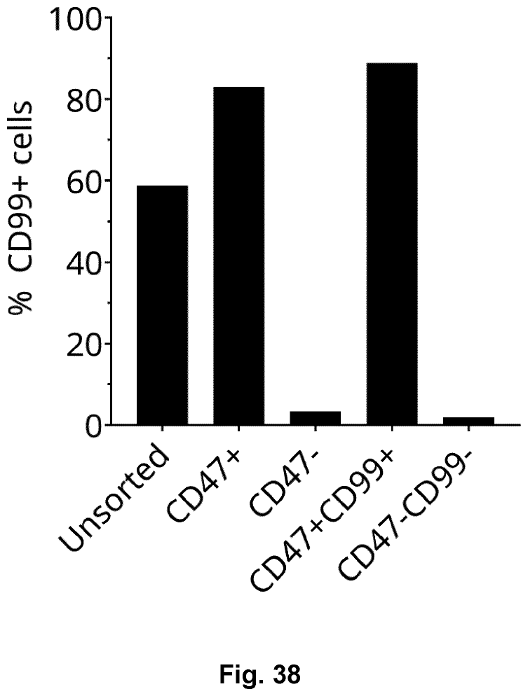

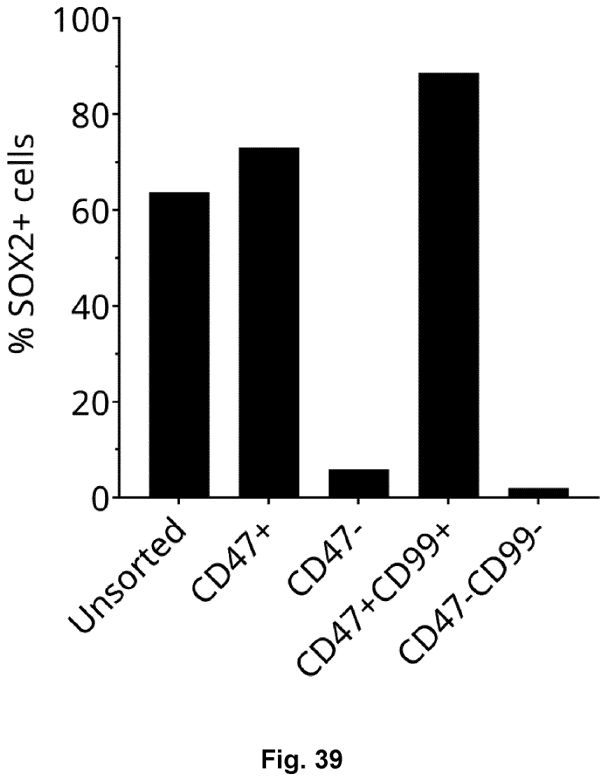

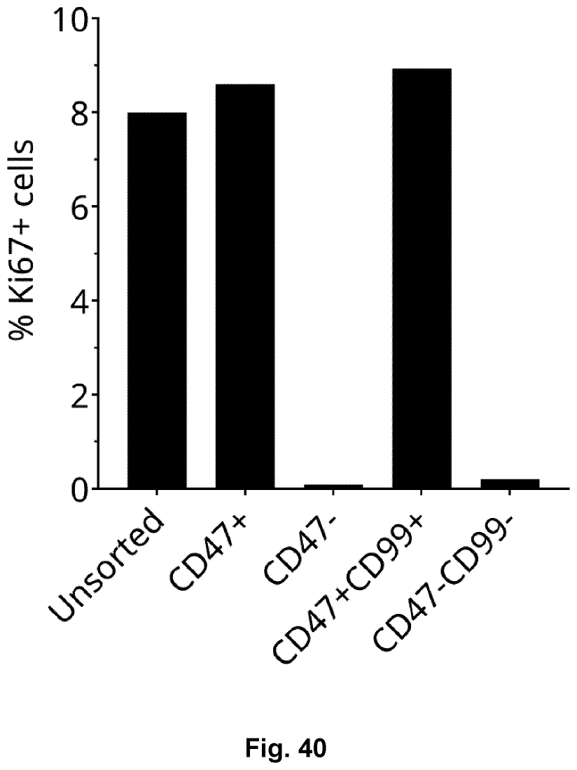

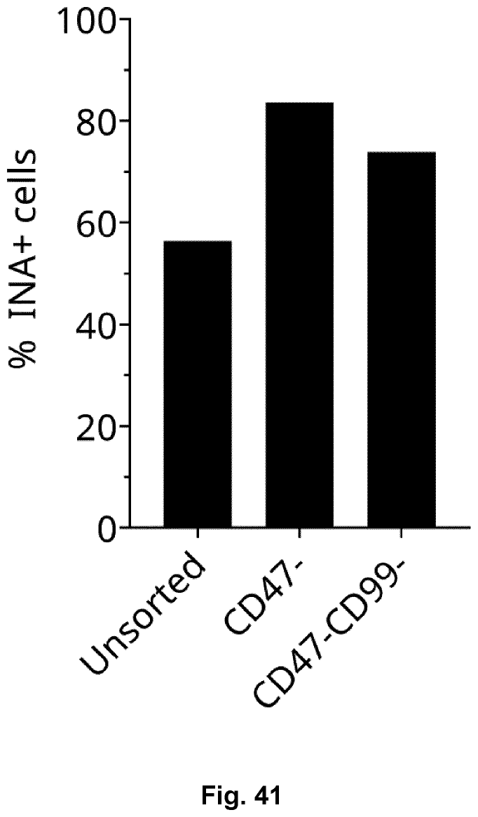

While FACS enrichment of CD47 negative or CD99 negative cells allowed enrichment of neuronal cells and a substantial reduction in the number of proliferative NSC/non-neuronal cell types, neither CD47 nor CD99 alone allowed complete depletion of these unwanted proliferative cell types. Interestingly, flow cytometry analysis of day in vitro (DIV) 25 vmDA cultures revealed that while a large proportion (34.5%) of the cells expressed both CD47 and CD99 at this stage (FIG. 34 Q2), there were also populations of cells expressing only CD47 (8.28%, FIG. 34 Q3) or CD99 (6.42%, FIG. 34 Q1), suggesting that the combination of these markers for enrichment would yield an even more pure neuronal population than when used on their own. In one experiment day 25 vmDA cultures were dissociated into a single cell suspension, stained with an antibody against CD47 either alone or in combination with an antibody against CD99, and the positive (CD47+) and negative (CD47−) or double positive (CD47+CD99+) and double negative (CD47−CD99−) cell populations, respectively, were purified by FACS and analysed by intracellular flow cytometry as previously described. First, the percentage of cells expressing CD47 and/or CD99 in the isolated fractions was determined. The unsorted control sample showed expression of CD47 and CD99 in about 60% of the cells (FIG. 37, 38). As expected, the percentage of cells expressing CD47 was increased in the CD47+ and CD47+CD99+ samples (>94%, FIG. 37) and almost entirely depleted in the CD47− and CD47−CD99− samples (<2.5%, FIG. 37), confirming successful separation of the populations. Similarly, the percentage of cells expressing CD99 was increased in both the CD47+ and CD47+CD99+ samples (>83%, FIG. 38) and almost entirely depleted in the CD47− and CD47−CD99− samples (<3.5%, FIG. 38), showing that enrichment of CD47− cells by CD47 antibody-based FACS results in a depletion of CD99+ cells. Both the CD47− and the CD47−CD99− fractions show markedly reduced proportions of cells expressing SOX2 (FIG. 39) and Ki67 (FIG. 40) and increased levels of neurons as marked by INA (FIG. 41)

TABLE 1
List of reagents used for FACS experiment

| Reagent | Supplier | Catalog number |
|---|---|---|
| CTS ™ Neurobasal ™ medium | Thermo Fisher Scientific | A1371201 |
| CTS ™ N-2 supplement | Thermo Fisher Scientific | A1370701 |
| B-27 ™ Supplement, XenoFree, minus | Thermo Fisher Scientific | A3353501 |
| vitamin A | | |
| GlutaMAX ™ Supplement | Thermo Fisher Scientific | 35050061 |
| Penicillin-Streptomycin | Thermo Fisher Scientific | 15140122 |
| LIVE/DEAD ™ Fixable Near-IR Dead Cell | Thermo Fisher Scientific | L10119 |
| Stain Kit | | |
| Y-27632 (ROCK inhibitor) | Miltenyi Biotec | 130-106-538 |
| Human serum albumin | Albumedix Ltd | 205-005 |
| DPBS without Ca2+ and Mg2+ | Thermo Fisher Scientific | 14190250 |
| DPBS with Ca2+ and Mg2+ | Thermo Fisher Scientific | 14040091 |
| StemPro ™ Accutase ™ | Thermo Fisher Scientific | A1110501 |
| Laminin-521 | Biolamina | LN521-05 |
| Poly-L-ornithine | Sigma-Aldrich | P4957 |
| BDNF | R&D Systems | 248-GMP |
| GDNF | R&D Systems | 212-GD |
| Dibutyryl cyclic-AMP (dcAMP) | Sigma-Aldrich | D0627 |
| L-Ascorbic acid | Sigma-Aldrich | A4403 |
| DAPT | Tocris Bioscience | 2634 |
| Transcription Factor Buffer Set | BD Biosciences | 562574 |
| Bovine Serum Albumin | Miltenyi Biotec | 130-091-376 |
| Distilled H2O | Thermo Fisher Scientific | 10977035 |
| CD47-APC antibody (clone: REA220) | Miltenyi Biotec | 130-123-254 |
| CD99-PE antibody (clone: 3B2/TA8) | Miltenyi Biotec | 130-123-763 |
| FOXA2-PE antibody (clone: REA506) | Miltenyi Biotec | 130-107-826 |
| SOX2-V450 antibody (clone: O30-678) | BD Biosciences | 561610 |
| Ki67-AF488 antibody (clone: B56) | BD Biosciences | 558616 |
| INA antibody | Atlas Antibodies | HPA008057 |
| SOX2 antibody | R&D Systems | AF2018 |
| FOXA2 antibody | Santa Cruz | Sc-374376 |
| Ki67 antibody | Invitrogen | MA5-14520 |
| TH antibody | Pel-Freez | P40101 |
| Donkey anti-Rabbit IgG AF488 | Invitrogen | A-21206 |
| Donkey anti-Rabbit IgG AF647 | Invitrogen | A-31573 |
| Donkey anti-Mouse IgG AF555 | Invitrogen | A-31570 |

### Example 6: Magnetic-Activated Cell Sorting (MACS) of Midbrain Neural Cells Using Magnetic Sorting an Antibody Against CD47

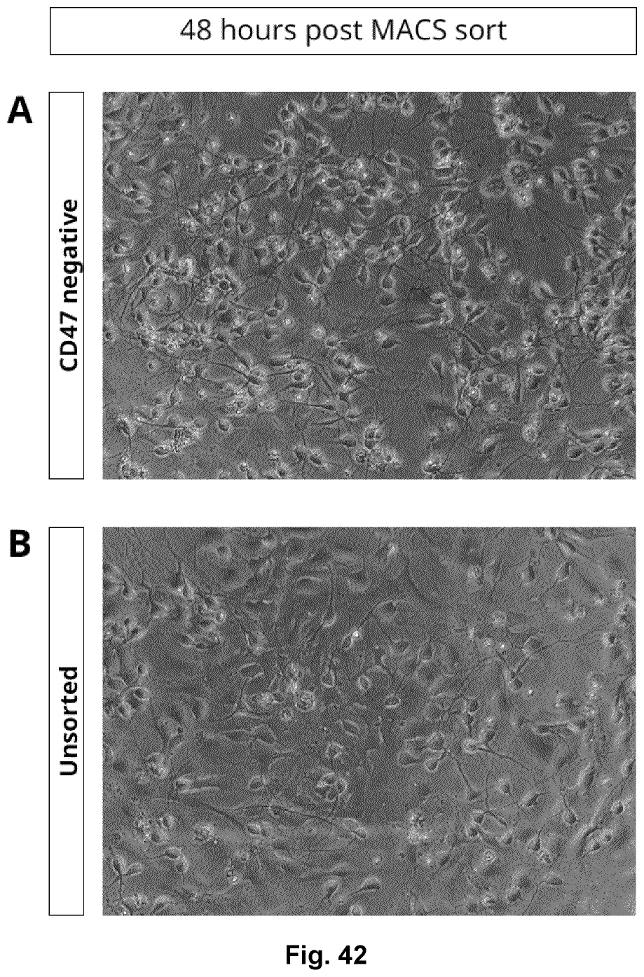

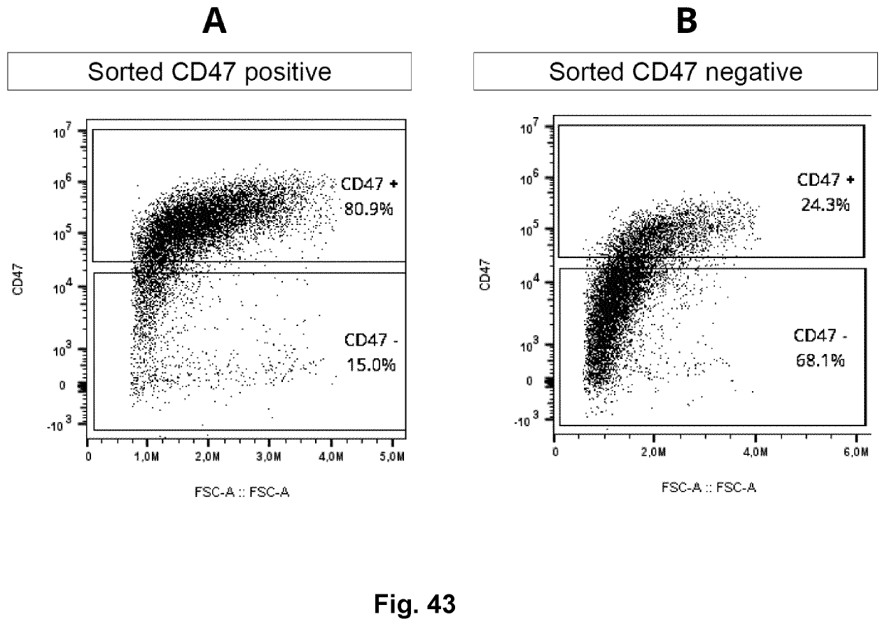

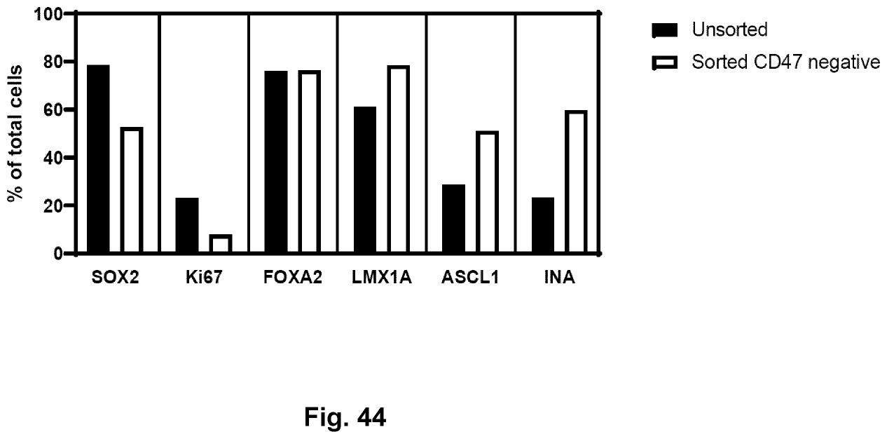

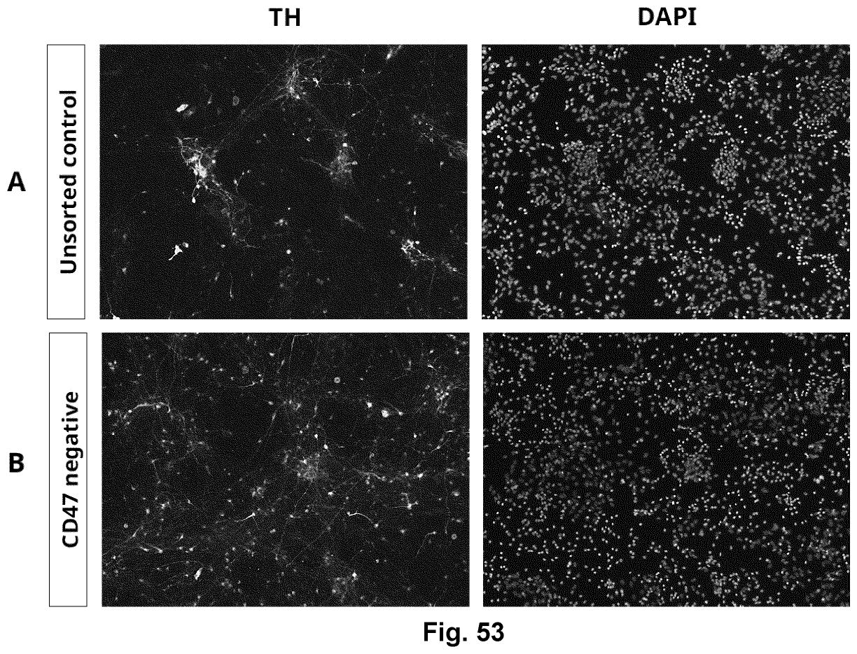

Ventral midbrain dopaminergic (vmDA) progenitor cells were generated from hESCs in 2D in vitro culture as described in example 1. At 24 days after initiating the differentiation, the cell culture was dissociated into a single cell suspension using accutase and collected in B27 media (CTS™ Neurobasal™ medium supplemented with 1% B-27™ supplement without vitamin A, 2 mM GlutaMAX™, 60 U/mL Penicillin-Streptomycin, 10 μM ROCK inhibitor). The cells were then resuspended in Hank's Balanced Salt Solution with 0.5% BSA, stained using an APC-conjugated antibody against CD47 at a concentration of 1:50 and subsequently labeled with anti-APC MicroBeads. CD47 positive and negative cells were purified by magnetic-activated cell sorting (MACS) using a LS MACS Column. The run through fraction containing unlabeled cells was collected. The magnetically retained cells were eluted and collected as positively selected fraction. After the MACS, the viability was confirmed to be >90% for both the CD47 negative and positive cell fraction. Immediately after the MACS, the sorted cells were re-seeded in 96-well plates coated with poly-L-ornithine (0.002%) and Laminin-521 (1.5 μg/cm²) in B27 media supplemented with BDNF (20 ng/mL), GDNF (20 ng/mL), L-ascorbic acid (200 μM), dcAMP (500 μM), DAPT (10 μM), and Y-27632 (10 μM) at a density of 200,000 cells/cm². Unsorted cells were used as control. Phase-contrast images acquired after 48 hours (FIG. 42) showed high viability of the sorted cells and clear differences in morphology between the unsorted and CD47 negative cells; the CD47 negative cells display typical neuronal morphology characterized by small, high-contrast, round/oval cell bodies with well-defined projections (FIG. 42 A), while the unsorted cells appear to have a mixture of neuronal cells and cells with large, low-contrast cell bodies with few or no projections (FIG. 42 B). Furthermore, immediately after cell sorting, samples of the sorted cells were processed for intracellular flow cytometry using the BD Transcription Factor Buffer Set, stained with antibodies against SOX2 (1:130), Ki-67 (1:2500), FOXA2 (1:320), LMX1A (1:2500), ASCL1 (1:2500) and INA (1:3000) recorded using a CytoFLEX™ flow cytometer, and analyzed using FlowJo 10.7.2 software. Firstly, the purity of the sorted samples was confirmed to be >68% (FIG. 43). The CD47 negative fraction was found to express lower levels of the neural stem cell marker SOX2 (52.7%; FIG. 44) as well as the proliferation marker Ki-67 (7.9%; FIG. 44), while the unsorted control showed higher levels of both SOX2 (78.6%; FIG. 44) and Ki-67 (23.1%; FIG. 44), corresponding to a difference in the percentage of cells expressing SOX2 and Ki67 of 33% and 65.8%, respectively. Furthermore, the CD47 negative faction was found to maintain equal numbers of cells expressing FOXA2, a marker of the developing floor plate (76%; FIG. 44) compared to the unsorted control. The CD47 negative fraction was also found to be enriched in cells expressing the early ventral midbrain marker LMX1A (78%; FIG. 44), the intermediate progenitor marker ASCL1 (51.2%; FIG. 44) and the neuron marker INA (59.8%; FIG. 44), while the unsorted control showed lower levels of all three markers (LMX1A 61%; ASCL1 28.8%; INA 23.3%; FIG. 44), corresponding to a difference in percentage of cells expressing LMX1A, ASCL1 and INA of 21.8%, 43.8% and 61%, respectively. These results show that CD47 antibody-based selection can be used for enrichment of neurons and depletion of proliferative neural stem cells and their derivatives. These results were supported by immunofluorescence (IF) staining of the cells re-seeded in vitro. At 10 days post-sort, the cells were fixed in 4% paraformaldehyde and stained with a primary antibody against the dopaminergic neuron marker tyrosine hydroxylase (TH; 1:500) followed by a fluorescently labelled secondary antibody. Images were acquired on a Zeiss Axio Observer microscope using ZEN 3.2 Pro software. The IF staining confirmed that enrichment of CD47 negative cells yields a population of cells enriched in dopaminergic neurons (FIG. 53).

TABLE 2
List of reagents used for MACS experiment

| Reagent | Supplier | Catalog number |
|---|---|---|
| CTS ™ Neurobasal ™ medium | Thermo Fisher Scientific | A1371201 |
| CTS ™ N-2 supplement | Thermo Fisher Scientific | A1370701 |
| B-27 ™ Supplement, XenoFree, minus vitamin A | Thermo Fisher Scientific | A3353501 |
| GlutaMAX ™ Supplement | Thermo Fisher Scientific | 35050061 |
| Penicillin-Streptomycin | Thermo Fisher Scientific | 15140122 |
| LIVE/DEAD ™ Fixable Near-IR Dead Cell Stain Kit | Thermo Fisher Scientific | L10119 |
| Y-27632 (ROCK inhibitor) | Miltenyi Biotec | 130-106-538 |
| Human serum albumin | Albumedix Ltd | 205-005 |
| DPBS without Ca²⁺ and Mg²⁺ | Thermo Fisher Scientific | 14190250 |
| DPBS with Ca²⁺ and Mg²⁺ | Thermo Fisher Scientific | 14040091 |
| StemPro ™ Accutase ™ | Thermo Fisher Scientific | A1110501 |
| Laminin-521 | Biolamina | LN521-05 |
| Poly-L-ornithine | Sigma-Aldrich | P4957 |
| BDNF | R&D Systems | 248-GMP |
| GDNF | R&D Systems | 212-GD |
| Dibutyryl cyclic-AMP (dcAMP) | Sigma-Aldrich | D0627 |
| L-Ascorbic acid | Sigma-Aldrich | A4403 |
| DAPT | Tocris Bioscience | 2634 |
| Transcription Factor Buffer Set | BD Biosciences | 562574 |
| Bovine Serum Albumin | Miltenyi Biotec | 130-091-376 |
| Distilled H₂O | Thermo Fisher Scientific | 10977035 |
| CD47-APC antibody (clone: REA220) | Miltenyi Biotec | 130-123-254 |
| CD99-PE antibody (clone: 3B2/TA8) | Miltenyi Biotec | 130-123-763 |
| FOXA2-PE antibody (clone: REA506) | Miltenyi Biotec | 130-107-826 |
| SOX2-V450 antibody (clone: O30-678) | BD Biosciences | 561610 |
| Ki67-AF488 antibody (clone: B56) | BD Biosciences | 558616 |
| TH antibody | Pel-Freez | P40101 |
| Donkey anti-Mouse IgG AF555 | Invitrogen | A-31570 |
| Anti-APC MicroBeads | Miltenyi Biotec | 130-090-855 |
| LS Columns | Miltenyi Biotec | 130-042-401 |

### Example 7: Differentiation of Human Pluripotent Stem Cells to Forebrain Neural Cells and Measurement of CD47 and CD99 Expression in these Cells

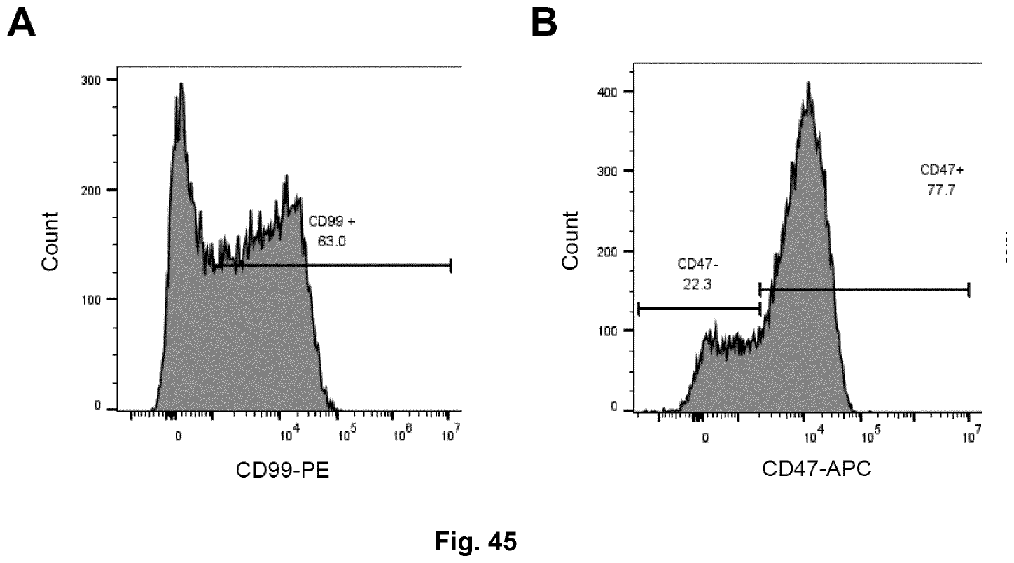

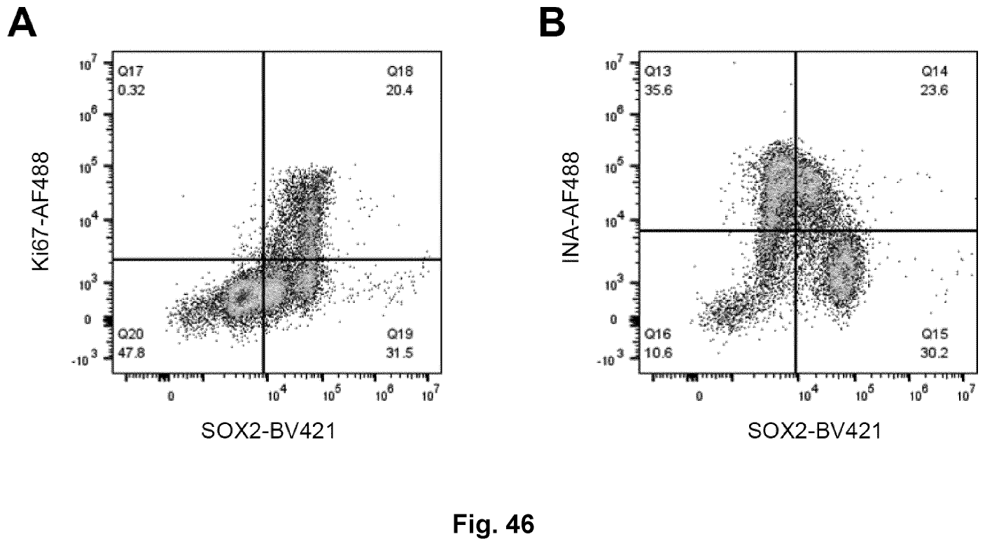

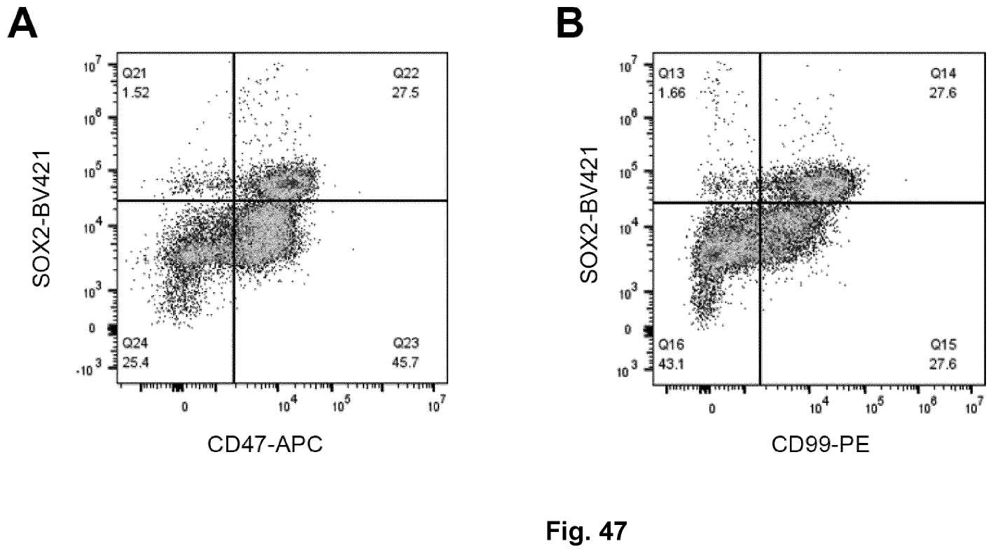

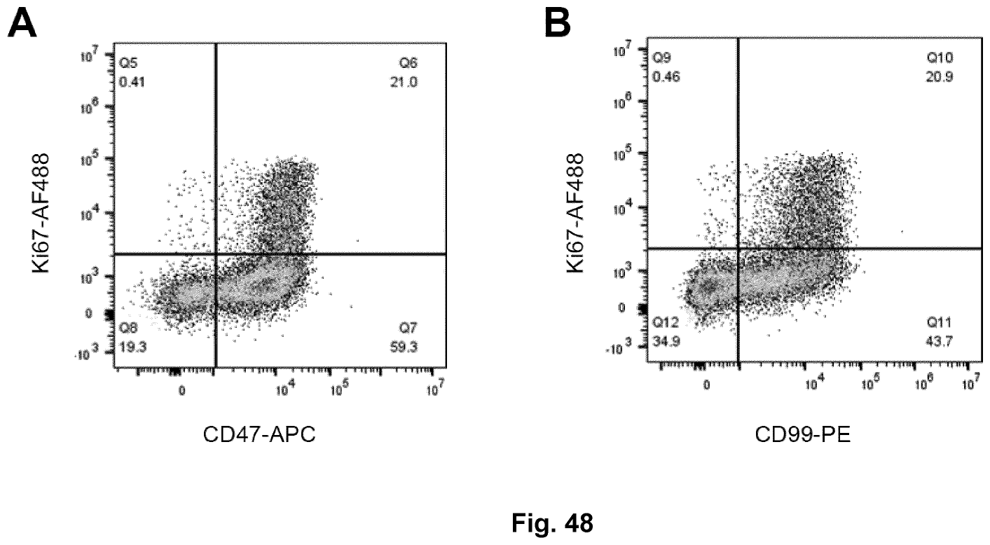

hESCs were differentiated according to an established protocol (Shi et al., 2012 (a); Shi et al., 2012 (b)). The hESCs were seeded and cultured on laminin-521 (1.2 μg/cm²; BioLamina) coated cultureware, and once forming a 95-100% confluent monolayer were exposed to differentiation media. From DIV0-10, the cells were cultured in an N2/1B27-based media: 50% DMEM/F12+Glutamax (Gibco) 50% Neurobasal (Gibco), 2% B27 supplement with vitamin A CTS (Thermo Fisher), 1% N2 supplement CTS (Thermo Fisher), 5% GlutaMAX (Thermo Fisher), 0.2% Penicillin streptomycin (P/S; Thermo Fisher), 1% NEAA (Gibco), 0,089% β-Mercaptoethanol (Gibco), supplemented with BMP pathway inhibitors LDN-193189 (100 nM; Miltenyi Biotec) and Noggin (100 ng/mL; Miltenyi Biotec) for neural induction. On DIV11, the cells were dissociated with 0.5 mM EDTA and passaged at a 1:2 ratio. N2/B27-based media was supplemented with fibroblast growth factor b (20 ng/mL; R&D) from DIV11-18. On DIV18, the cells were dissociated with 0.5 mM EDTA and either cryopreserved or passaged at a 1:2 ratio. From DIV20 onwards, the cells were cultured in N2/B27-based media without supplements and passaged on DIV32. At DIV35 the forebrain neural cells were dissociated into a single cell suspension using accutase, stained with fluorophore-conjugated antibodies against CD47 and CD99, and analyzed by flow cytometry as described in example 5. At this stage of the differentiation, approximately 50% of the cells expressed the neural stem cell marker SOX2 (FIG. 46), while approximately 20% expressed the proliferation markers Ki67 (FIG. 46 A), indicating these cells are still mitotic neural stem cells, and approximately 60% expressed the neuronal marker INA (FIG. 46 B), indicating these have differentiated or are in the process of differentiating into neurons. The analysis showed that 63% of the cells expressed CD99 (FIG. 45 A) and 78% expressed CD47 (FIG. 45 B). Furthermore, it was found that the vast majority of cells expressing CD99 and CD47 co-expressed both SOX2 (FIG. 47) and Ki67 (FIG. 48). These results strongly support the hypothesis that CD99 and CD47 can be used as antibody targets for enrichment of forebrain neurons and depletion of proliferative cells by FACS or MACS.

### REFERENCES

Shi Y, Kirwan P, Livesey F J. Directed differentiation of human pluripotent stem cells to cerebral cortex neurons and neural networks. Nat Protoc. 2012 October; 7(10):1836-46. doi: 10.1038/nprot.2012.116. Epub 2012 Sep. 13. PMID: 22976355. (a)
Shi Y, Kirwan P, Smith J, Robinson H P, Livesey F J. Human cerebral cortex development from pluripotent stem cells to functional excitatory synapses. Nat Neurosci. 2012 Feb. 5; 15(3):477-86, S1. doi: 10.1038/nn.3041. PMID: 22306606; PMCID: PMC3882590. (b)

### Example 8: Differentiation of Human Pluripotent Stem Cells to Hindbrain/Spinal Cord Neural Cells and Measurement of CD47 and CD99 Expression in these Cells

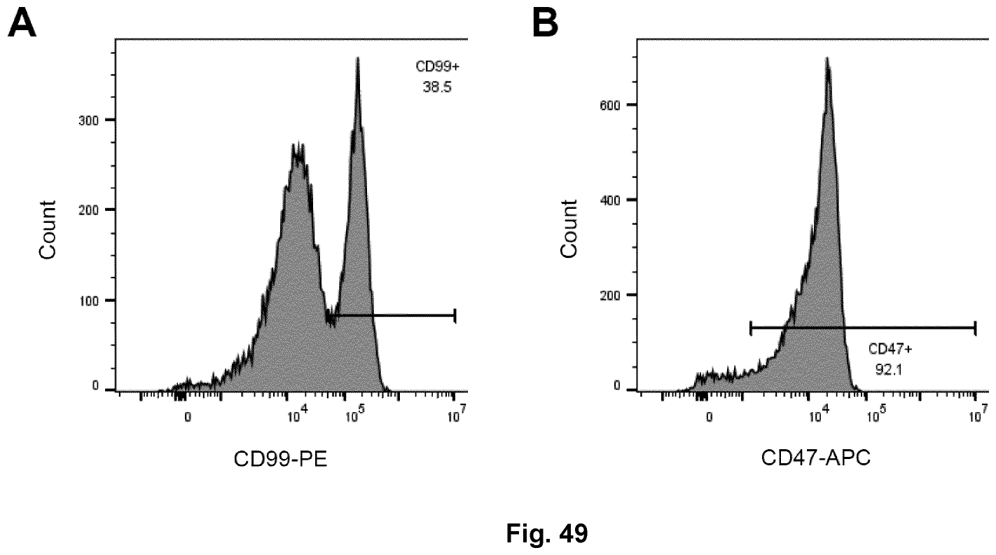

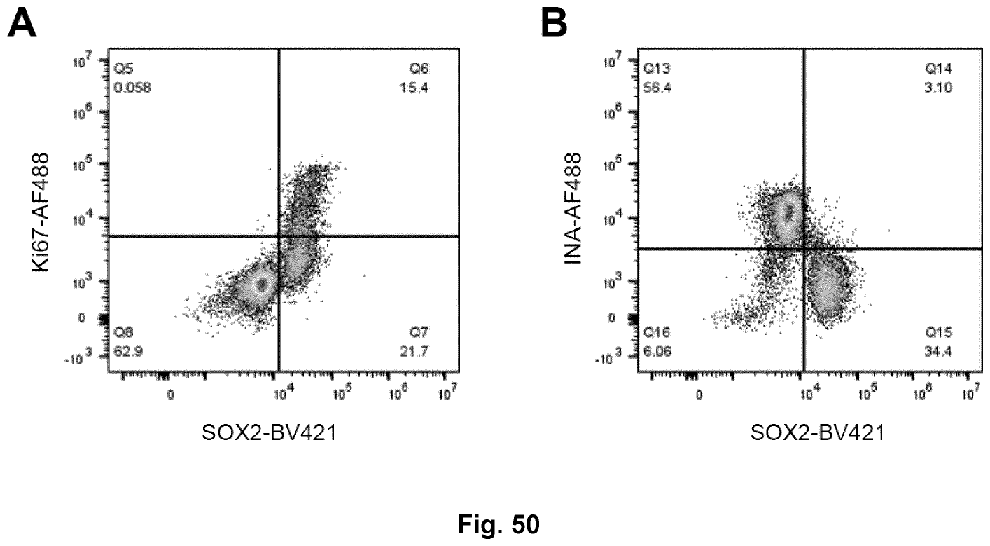

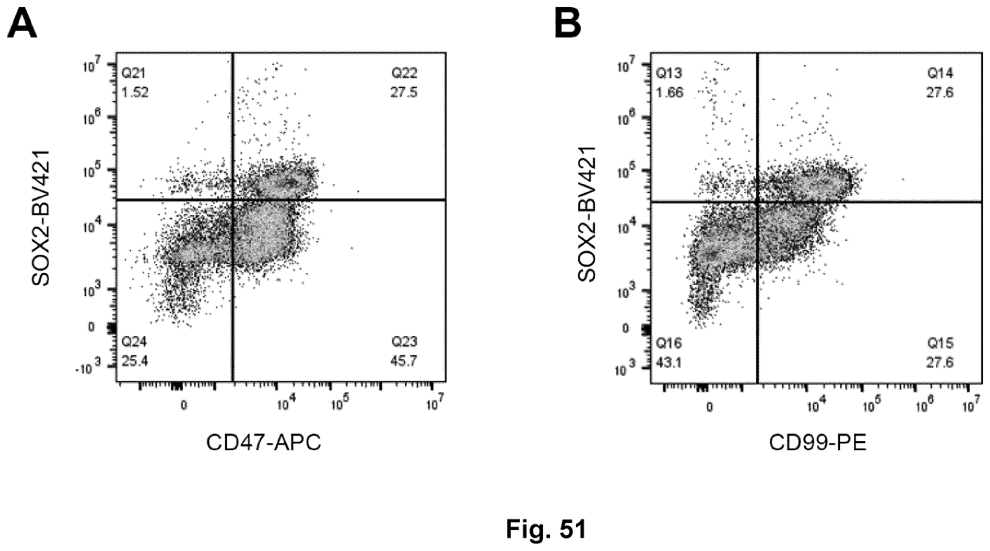

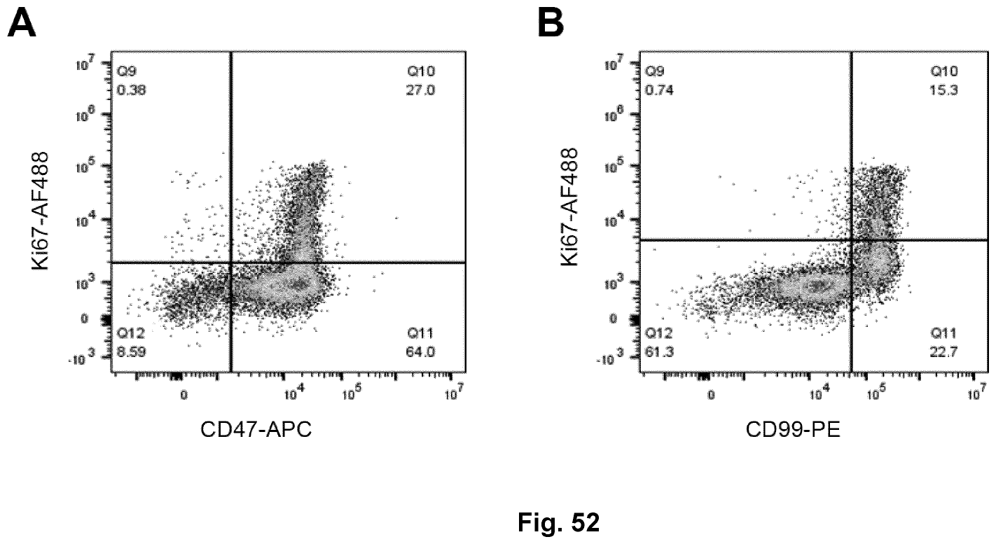

Undifferentiated hESCs at 80-90% confluence were disassociated with EDTA 0.5 mM (Thermo Fisher) for 5-7 min at room temperature to detach from flasks for re-seeding. Aggregates of hESCs were seeded onto Laminin-521 (1.2 μg/cm²) pre-coated 24-well plates or T25 flasks (Sarstedt) and distributed evenly. The first 24 hours, cells were maintained in PS Brew XF (Miltenyi Biotec) and supplemented with 10 μM Y27632 (ROCKi; Miltenyi Biotec) to enhance cell survival. Media was changed every 24 hours and cells were grown to 90-100% confluence at which point differentiation was initiated. For the first 6 days of differentiation, cells were maintained in Pan-Neuronal Base Media consisting of 45% Neurobasal (Gibco), 45% DMEM/F12+GlutaMAX (Gibco), 5% B27 supplement (Thermo Fisher), 1% N2 supplement (Thermo Fisher), 1% MEM Non-essential amino acids (NEAA; Gibco), 0.5% GlutaMAX (Gibco), 0.2% Penicillin/streptomycin (P/S; Thermo Fisher), supplemented with BMP pathway inhibitors LDN-193189 (100 nM; Miltenyi Biotec) and Noggin (100 ng/mL; Miltenyi Biotec) for neural induction. 3 μM CHIR99021 (CHIR; Miltenyi Biotec) was added for caudalization, and the media was changed daily. At day 6, the cells were dissociated using accutase and passaged at a 1:6 ratio. From DIV6-12, cells were grown in Pan-Neuronal Base Media supplemented with BMP pathway inhibitors LDN-193189 (100 nM; Miltenyi Biotec) and Noggin (100 ng/mL; Miltenyi Biotec), plus 1 μM CHIR, 100 nM Purmorphamine (PM; Tocris) and 500 nM Retinoic acid (RA; Sigma). From days 12 to 18, cells were grown in Pan-Neuronal Base Media (without vitamin A) supplemented with 100 nM PM and 500 nM RA. At DIV15, cells were dissociated using accutase and passaged at a 1:6 ratio. From DIV18 onwards cultures were maintained in pan neuronal base media (-VitA) for maturation and to promote neuron formation. Media was changed daily and cells were washed with DPBS−/− prior to media change to remove cell death. On DIV24, the hindbrain/spinal cord neural cells were dissociated into a single cell suspension using accutase, stained with fluorophore-conjugated antibodies against CD47 and CD99, and analyzed by flow cytometry as described in example 5. At this stage of the differentiation, approximately 37% of the cells expressed the neural stem cell marker SOX2 (FIG. 50), while approximately 15% expressed the proliferation markers Ki67 (FIG. 50 A), indicating these cells are still mitotic neural stem cells, and approximately 60% expressed the neuronal marker INA (FIG. 50 B), indicating these have differentiated or are in the process of differentiating into neurons. The analysis showed that 39% of the cells expressed CD99 (FIG. 49 A) and 92% expressed CD47 (FIG. 49 B). Furthermore, it was found that the vast majority of cells expressing CD99 and CD47 co-expressed both SOX2 (FIG. 51) and Ki67 (FIG. 52). These results strongly support the hypothesis that CD99 and CD47 can be used as antibody targets for enrichment of hindbrain/spinal cord neurons and depletion of proliferative cells by FACS or MACS.

### Example 9: RNA Sequencing Methodology and Experimental Design to Identify Markers for Enrichment or Depletion

An in vitro time series of hPSC-derived ventral midbrain neural cells was sampled with single cell RNA sequencing in order to interrogate the transcriptomic signature of heterogeneous hPSC-derived ventral midbrain cell cultures (FIG. 1 and Example 2) for the purpose of identifying in an unbiased manner genes unique to or upregulated in desired cell populations (i.e. ventral midbrain intermediate precursors/neuroblasts marked by ASCL1) and undesired populations (i.e. non-ventral midbrain floorplate identity cells, non-neuronal precursors, non-neuronal cell types).

Desired and undesired populations (or clusters as described in the field of single cell RNA sequencing and transcriptomics) were identified in sequencing datasets (i.e. tSNE plots shown in FIG. 5) based on gene expression profiles of known genes. Comparisons were made between these clusters to identify novel genes/markers of desired or undesired populations.

To perform single cell RNA sequencing (scRNA-seq), cell clusters of undifferentiated PSCs as well as those of differentiated cells were dissociated into single cell suspensions with accutase, tryple select or other such reagents and 3000-10000 cells were processed using the 10X Genomics Chromium Platform and sequenced on a NextSeq550. Data was processed using 10X cellranger and the Seurat analysis package in R programming language. Samples were analysed, filtered for low quality or multiplet cells and analyzed separately for each individual experiment before combining the cells of the selected differentiated cell lineages of choice as well as the hPSCs into one dataset that were then analysed using the standard Seurat workflow as outlined for Seurat version 3, i.e. normalizing using SCTransform and finally using the first 29 principal components for the unified tSNE plots (such as those shown in FIG. 5).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for providing a cell population enriched in neurons and/or precursors thereof, comprising the steps of:
obtaining a cell population comprising neuron precursors,
selecting cells positive for one or more of the markers selected from PTPRO, ADGRG1, EPHB2, DCC, DLL3, DLL1, ADCYAP1, GPM6A, GAP43, C1QL1, NRXN1, LRRC4C, TNFRSF21, FZD1, FREM2, TRPM8, CACNA2D1, CLDN5, CHRNA5, and GPR85, and/or excluding cells positive for one or more of the markers selected from SLIT2, NTN1, CMTM6, CMTM8, CMTM7, CRLF1, CD36, CD47, CD99, CD83, TNFRSF1A/CD120a, TNFRSF12a/CD266, TMEM123, LRP10, ITGB8, HLA-A, and RAMP1.

2. The method according to claim 1, wherein the cell population comprising neuron precursors is obtained by the steps of obtaining a cell population comprising neural stem cells, wherein at least 80% of the cell population comprising neural stem cells express the marker SOX2, and culturing the cell population comprising neural stem cells until the expression of SOX2 has decreased by at least 5%.

3. The method according to claim 2, wherein the cell population comprises neural stem cells of the ventral midbrain region, and wherein the cells positive for the marker CD47 are excluded.

4. The method according to claim 2, wherein the cell population comprises neural stem cells of the forebrain, midbrain, hindbrain, or spinal cord region, and wherein cells positive for one or both of the markers CD99 and CD47 are excluded.

5. The method according to claim 1, wherein 10-90% of the cell population are positive for the marker SOX2, 5-50% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1, and 5-20% of the cell population are positive for one or more of the markers INA, TUBB3, STMN2, MAP2, and RBFOX3, when selecting cells.

6. The method according to claim 3, wherein 10-90% of the cell population are positive for the marker SOX2, 5-50% of the cell population are positive for one or more of the markers ASCL1, and 5-20% of the cell population are positive for one or more of the markers INA, when selecting and/or excluding cells.

7. The method according to claim 1, wherein obtaining the cell population comprising neuron precursors comprises the step of differentiating PSCs into neural stem cells.

8. The method according to claim 7, wherein the PSCs are allowed to differentiate into neural stem cells for at least 20 days prior to the step of selecting cells.

9. The method according to claim 1, wherein the step of selecting cells is carried out by isolating cells using MACS.

10. An in vitro cell population of neurons and/or precursors thereof, obtained by the method according to claim 1.

11. An in vitro cell population of neurons and/or precursors thereof, wherein less than 5% of the cell population are positive for one or more of the markers selected from SLIT2, NTN1, CMTM6, CMTM8, CMTM7, CRLF1, CD36, CD47, CD99, CD83, TNFRSF1A/CD120a, TNFRSF12a/CD266, TMEM123, LRP10, ITGB8, HLA-A, and RAMP1.

12. The cell population according to claim 11, wherein less than 5% of the cell population are positive for the marker CD99.

13. The cell population according to claim 11, wherein less than 5% of the cell population are positive for the marker CD47 and at least 80% of the cell population are positive for the marker FOXA2.

14. The cell population according to claim 11, wherein at least 80% of the cell population are ventral midbrain neurons and/or precursors thereof.

15. The cell population according to claim 12, wherein at least 80% of the cell population are forebrain, hindbrain, or spinal cord neurons and/or precursors thereof.

16. The cell population according to claim 12, wherein at least 80% of the cell population are ventral midbrain neurons and/or precursors thereof.

17. The cell population according to claim 13, wherein at least 80% of the cell population are ventral midbrain neurons and/or precursors thereof.

18. The method according to claim 3, wherein 10-90% of the cell population are positive for the marker SOX2, 5-50% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1, and 5-20% of the cell population are positive for one or more of the markers INA, TUBB3, STMN2, MAP2, and RBFOX3, when selecting and/or excluding cells.

19. The method according to claim 4, wherein 10-90% of the cell population are positive for the marker SOX2, 5-50% of the cell population are positive for one or more of the markers ASCL1, NEUROD1, NEUROD2, NEUROD4, NEUROD6, NEUROG1, NEUROG2, NEUROG3, EOMES, CHX10, ISL1, SOX4, SOX11, NKX2.1, NFE2L2, and NHLH1, and 5-20% of the cell population are positive for one or more of the markers INA, TUBB3, STMN2, MAP2, and RBFOX3, when selecting and/or excluding cells.

20. The method according to claim 3, wherein the step of selecting and/or excluding cells is carried out by isolating cells using MACS.

21. The method according to claim 4, wherein the step of selecting and/or excluding cells is carried out by isolating cells using MACS.
